(19) United States
(12) Patent Application Publication
(10) Pub. No.: US 2018/0362944 A1
(43) Pub. Date: Dec. 20, 2018

(54) CONDITIONAL GUIDE RNAs

(71) Applicant: California Institute of Technology, Pasadena, CA (US)

(72) Inventors: Mikhail H Hanewich-Hollatz, Pasadena, CA (US); Zhewei Chen, Pasadena, CA (US); Lisa Hochrein, Pasadena, CA (US); Jining Huang, Pasadena, CA (US); Niles A. Pierce, Pasadena, CA (US)

(21) Appl. No.: 16/005,445

(22) Filed: Jun. 11, 2018

Related U.S. Application Data
(60) Provisional application No. 62/518,442, filed on Jun. 12, 2017.

(51) Int. Cl. C12N 9/22; C12N 15/113; C12N 15/85; C12N 15/90; C12P 19/34

(52) U.S. Cl. CPC C12N 9/22; C12N 15/1131; C12P 19/34; C12N 15/907; C12N 15/85

(57) ABSTRACT

Described herein are conditional guide RNAs that change their activity status depending on the presence or absence of an input target, forming a complex with an RNA-guided effector and conditionally performing a downstream function on a target nucleic acid. Methods for conditionally performing a downstream function on the target nucleic acid using conditional guide RNAs are also described.

### CROSS-REFERENCE TO RELATED APPLICATIONS

This application claims the benefit of U.S. Provisional Application 62/518,442 filed on Jun. 12, 2017, which is hereby incorporated by reference in its entirety.

### STATEMENT REGARDING FEDERALLY SPONSORED R&D

This invention was made with government support DARPA (HR0011-17-2-0008). The government has certain rights in the invention.

### REFERENCE TO ELECTRONIC SEQUENCE LISTING

The present application is being filed along with an Electronic Sequence Listing. The Electronic Sequence Listing is provided as a file entitled CALTE131ASEQLIST.txt which is 9,415 bytes in size, created on Jun. 11, 2018. The information in the Electronic Sequence Listing is incorporated herein by reference in its entirety.

### BACKGROUND

### Field

The disclosure is generally related to guide RNAs that function in a conditional manner.

### Description of the Related Art

The CRISPR/Cas system exists in nature as a prokaryotic immune system, enabling nucleic acid sequence-specific acquired immunity to foreign genetic elements (Barrangou et al. 2007; Horvath and Barrangou 2010). Recent developments in the engineering and implementation of RNA-guided CRISPR effectors allow for the high-fidelity sequence-specific interaction of CRISPR effectors with target nucleic acids in a variety of organisms and settings for a number of applications (Sander and Joung 2014).

### SUMMARY

In some embodiments, a conditional guide RNA (cgRNA) is provided. The cgRNA is configured to change its activity status depending upon a presence or an absence of an input target. In some embodiments, the cgRNA further forms a complex with an RNA-guided effector, such that the complex is configured to bind to a specific target nucleic acid.

In some embodiments, a cgRNA is provided that comprises an input target binding region, configured to bind to an input target; a target binding region, configured to bind to a target nucleic acid; and an effector handle region. The cgRNA is configured to conditionally perform a downstream function on the target nucleic acid in a presence of the input target and an RNA-guided effector.

In some embodiments, a cgRNA is provided that comprises an input target binding region, configured to bind to an input target; a target binding region, configured to bind to a target nucleic acid; and an effector handle region. The cgRNA is configured to interact and form a complex with an RNA-guided effector, and the complex is configured to conditionally perform a downstream function on the target nucleic acid in an absence of the input target.

In some embodiments, a method is provided that comprises providing a conditional guide RNA, wherein the cgRNA changes its activity status depending upon a presence or an absence of an input target. In some embodiments, the method further comprises forming a complex with an RNA-guided effector and binding a specific target nucleic acid.

In some embodiments, the effector handle region is configured to interact and form a complex with an effector protein selected from the group consisting of Cas9, dCas9, C2C2, Cas13d, any protein fusions or derivatives thereof, any RNA-guided CRISPR effector protein or protein complex, or any protein from a similar pathway.

In some embodiments, a cgRNA is provided that comprises a target binding region, configured to bind to a target nucleic acid and an effector handle region. The cgRNA is configured to interact and form a complex with an RNA-guided effector, the cgRNA is configured to conditionally perform a downstream function on the target nucleic acid in an absence of an input target, the cgRNA comprises from 5′ to 3′ the target binding region comprising a domain a, the effector handle, and an optional terminator region, the cgRNA is configured to be active, and the cgRNA is inactivated by the binding of a domain a* of an input target and the domain a of the target binding region to each other.

In some embodiments, a conditional guide RNA (cgRNA) is provided. The cgRNA is configured to change its activity status depending upon a presence or an absence of an input target. In some embodiments, it further forms a complex with an RNA-guided effector, such that the complex is configured to bind to a specific target nucleic acid.

In some embodiments, a cgRNA is provided and comprises an input target binding region, configured to bind to an input target; a target binding region, configured to bind to a target nucleic acid; and an effector handle region. The cgRNA is configured to conditionally perform a downstream function on the target nucleic acid in a presence of the input target and an RNA-guided effector.

In some embodiments, a cgRNA is provided that comprises an input target binding region, configured to bind to an input target; a target binding region, configured to bind to a target nucleic acid; and an effector handle region. The cgRNA is configured to interact and form a complex with an RNA-guided effector. The complex is configured to conditionally perform a downstream function on the target nucleic acid in an absence of the input target.

In some embodiments, the cgRNA comprises from 5′ to 3′ the 5′ extension region comprising a domain a, a domain b, and a domain c, the target binding region comprising a domain b*, and a domain d, the effector handle region, wherein the domain b of the 5′ extension region and the domain b* of the target binding region are complementary to each other, and wherein the cgRNA is configured to be inactive by the binding of the domain b of the 5′ extension region and the domain b* of the target binding region to each other.

In some embodiments, the cgRNA comprises from 5′ to 3′ the 5′ extension region comprising a domain a, and a domain b, the target binding region, a first partial sequence of the effector handle, a modified effector handle loop region comprising a domain b*, a second partial sequence of the effector handle, and an optional terminator region, wherein the domain b of the 5′ extension region and the domain b* of the modified effector handle loop region are complementary to each other, and wherein the cgRNA is configured to be inactive by the binding of the domain b of the 5′ extension region and the domain b* of the modified effector handle loop region to each other.

In some embodiments, the cgRNA comprises from 5′ to 3′ the 5′ extension region comprising a domain a, a domain b, and a domain c, the target binding region comprising a domain d and a domain c*, the effector handle comprising a domain b*, and an optional terminator, wherein the domain b of the 5′ extension region and the domain b* of the effector handle are complementary to each other, wherein the domain c of the 5′ extension region and the domain c* of the target binding region are complementary to each other, and wherein the cgRNA is configured to be inactive by the binding of the domain b of the 5′ extension region and the domain b* of the effector handle to each other, and by the binding of the domain c of the 5′ extension region and the domain c* of the target binding region to each other.

In some embodiments, the cgRNA comprises from 5′ to 3′ the 5′ extension region comprising a domain a, and a domain b, the target binding region, the effector handle, a terminator insert region comprising domain b*, and a terminator region, wherein domain b of the 5′ extension region and the domain b* of the terminator insert region are complementary to each other, and wherein the cgRNA is configured to be inactive by the binding of domain b of the 5′ extension region and the domain b* of the terminator insert region to each other.

In some embodiments, the cgRNA comprises from 5′ to 3′ the target binding region, a first partial sequence of the effector handle, a modified effector handle loop region comprising a domain a, a domain b, and a domain c, a second partial sequence of the effector handle, a terminator insert region comprising domain b*, and a terminator region, wherein the domain b of the modified effector handle loop region and domain b* of the terminator insert region are complementary to each other, and wherein the cgRNA is configured to be inactive by the binding of the domain b of the modified effector handle loop region and domain b* of the terminator insert region to each other.

In some embodiments, the cgRNA comprises from 5′ to 3′ the 5′ extension region comprising a domain a, a domain c*, a domain b, and a domain c, the target binding region comprising a domain b* and a domain d, the effector handle, and an optional terminator region, wherein the domain b of the 5′ extension region and the domain b* of the target binding region are complementary to each other, and the domain c and the domain c* of the 5′ extension region are complementary to each other, and wherein the cgRNA is configured to be active by the binding of the domain c and the domain c* of the 5′ extension region to each other.

In some embodiments, the cgRNA comprises from 5′ to 3′ the 5′ extension region comprising a domain a, the target binding region, a first partial sequence of the effector handle, a modified effector handle loop region comprising a domain b, a second partial sequence of the effector handle, and an optional terminator region, wherein the cgRNA is configured to be active.

In some embodiments, the cgRNA comprises from 5′ to 3′ the target binding region, a first partial sequence of the effector handle, a modified effector handle loop region comprising a domain a, a second partial sequence of the effector handle, a terminator insert region comprising a domain b, and a terminator region, wherein the cgRNA is configured to be active.

In some embodiments, the cgRNA comprises from 5′ to 3′ the target binding region, the effector handle, a terminator insert region comprising a domain a, and a terminator region, wherein the cgRNA is configured to be active.

In some embodiments, the cgRNA comprises from 5′ to 3′ the target binding region, the effector handle, a first partial sequence of a terminator region, a modified terminator loop region comprising a domain a, and a second partial sequence of the terminator region, wherein the cgRNA is configured to be active.

In some embodiments, the input target comprises from 3′ to 5′ a domain a* and the domain b*, and wherein the domain a and the domain a* are complementary to each other, and the domain b and the domain b* are complementary to each other, and wherein the cgRNA is configured to be activated by the binding of the domain a of the 5′ extension region and domain a* (of the input target to each other and domain b of the 5′ extension region and the domain b* of the input target to each other.

In some embodiments, the input target comprises from 3′ to 5′ a domain a* and the domain b*, wherein the domain a and the domain a* are complementary to each other, and wherein the domain b and the domain b* are complementary to each other, and wherein the cgRNA is configured to be activated by the binding of the domain a of the 5′ extension region and domain a* of the input target to each other and domain b of the 5′ extension region and the domain b* of the input target to each other.

In some embodiments, the input target comprises from 3′ to 5′ a domain a*, the domain b*, and the domain c*, wherein the domain a and the domain a* are complementary to each other, and wherein the domain b and the domain b* are complementary to each other, and wherein the domain c and the domain c* are complementary to each other, and wherein the cgRNA is configured to be activated by the binding of the domain a of the 5′ extension region and domain a* of the input target to each other, domain b of the 5′ extension region and the domain b* of the input target to each other, and domain c of the 5′ extension region and the domain c* of the input target to each other.

In some embodiments, the input target comprises from 3′ to 5′ a domain a*, and the domain b*, wherein the domain a and the domain a* are complementary to each other, and wherein the domain b and the domain b* are complementary to each other, and wherein the cgRNA is configured to be activated by the binding of the domain a of the 5′ extension region and domain a* of the input target to each other and domain b of the 5′ extension region and the domain b* of the input target to each other.

In some embodiments, the input target comprises from 3′ to 5′ a domain a* and the domain b*, wherein the domain a and the domain a* are complementary to each other, and wherein the domain b and the domain b* are complementary to each other, and wherein the cgRNA is configured to be activated by the binding of the domain a of the modified effector handle loop region and domain a* of the input target to each other and domain b of the modified effector handle loop region and the domain b* of the input target to each other.

In some embodiments, the input target comprises from 3′ to 5′ a domain a* and domain c, wherein the domain a and the domain a* are complementary to each other, and domain c and the domain c* are complementary to each other, and wherein the cgRNA is configured to be inactivated by the binding of the domain a* of the input target and domain a of the 5′ extension region to each other and the domain c of the input target and domain c* of the 5′ extension region to each other.

In some embodiments, the input target comprises from 3′ to 5′ a domain a* and domain b*, wherein the domain a of the 5′ extension region and the domain a* of the input target are complementary to each other, and domain b of the modified effector handle loop region and the domain b* of the input target are complementary to each other, and wherein the cgRNA is configured to be inactivated by the binding of the domain a of the 5′ extension region and the domain a* of the input target to each other and the domain b of the modified effector handle loop region and the domain b* of the input target to each other.

In some embodiments, the input target comprises from 3′ to 5′ a domain a* and a domain b*, wherein the domain a of the modified effector handle loop region and the domain a* of the input target are complementary to each other, and domain b of the terminator insert region and the domain b* of the input target are complementary to each other, and wherein the cgRNA is configured to be inactivated by the binding of the domain a of the modified effector handle loop region and the domain a* of the input target to each other and the domain b of the terminator insert region and the domain b* of the input target to each other.

In some embodiments, the input target comprises from 3′ to 5′ a domain a*, and wherein the domain a of the terminator insert region and the domain a* of the input target are complementary to each other, and wherein the cgRNA is configured to be inactivated by the binding of the domain a of the terminator insert region and the domain a* of the input target to each other.

In some embodiments, the input target comprises from 3′ to 5′ a domain a*, and wherein the domain a of the modified terminator loop region and the domain a* of the input target are complementary to each other, and wherein the cgRNA is inactivated by the binding domain a of the modified terminator loop region and the domain a* of the input target to each other.

In some embodiments, a method is provided that comprises providing a conditional guide RNA, wherein the cgRNA changes its activity status depending upon a presence or an absence of an input target.

In some embodiments, the method further comprises forming a complex with an RNA-guided effector and binding a specific target nucleic acid.

In some embodiments, the method is for conditionally performing a downstream function on a target nucleic acid. The method comprises providing an inactive conditional guide RNA (cgRNA) comprising: an input target binding region, configured to bind to an input target; a target binding region, configured to bind to the target nucleic acid; and an effector handle region. The method further comprises conditionally performing a downstream function on the target nucleic acid by providing an input target and an RNA-guided effector. By a binding of the input target to the cgRNA, the cgRNA is activated to perform a downstream function on the target nucleic acid.

In some embodiments, the method comprises providing an inactive conditional guide RNA (cgRNA) according to any of the embodiments above, conditionally performing a downstream function on the target nucleic acid by providing an input target according to any of the embodiments above and an RNA-guided effector, wherein by a binding of the input target to the cgRNA, the cgRNA is activated to perform a downstream function on the target nucleic acid.

In some embodiments, the method comprises providing an inactive conditional guide RNA (cgRNA) according to any of the embodiments above and conditionally performing a downstream function on the target nucleic acid by providing an input target according to any of the embodiments above and an RNA-guided effector, wherein by a binding of the input target to the cgRNA, the cgRNA is activated to perform a downstream function on the target nucleic acid.

In some embodiments, the method comprises providing an inactive conditional guide RNA (cgRNA) according to any of the embodiments above and conditionally performing a downstream function on the target nucleic acid by providing an input target according to any of the embodiments above and an RNA-guided effector, wherein by a binding of the input target the cgRNA, the cgRNA is activated to perform a downstream function on the target nucleic acid.

In some embodiments, the method is for conditionally performing a downstream function on a target nucleic acid. The method comprises providing an active conditional guide RNA (cgRNA) comprising: an input target binding region, configured to bind to an input target; a target binding region, configured to bind to the target nucleic acid; and an effector handle region. The cgRNA is configured to interact and form a complex with an RNA-guided effector and conditionally performing a downstream function on the target nucleic acid by providing an input target. By a binding of the input target to the cgRNA, the cgRNA ceases to perform a downstream function on the target nucleic acid.

In some embodiments, the method comprises providing a conditional guide RNA (cgRNA) according to any of the embodiments above and conditionally performing a downstream function on the target nucleic acid by providing an input target according to any of the embodiments above, wherein by a binding of the input target to the cgRNA, the cgRNA ceases to perform a downstream function on the target nucleic acid.

In some embodiments of the method the downstream function is selected from the group consisting of activating an expression of the target nucleic acid, silencing an expression of the target nucleic acid, editing the target nucleic acid, and binding the target nucleic acid.

In some embodiments, of the method changing the activity status of the cgRNA results in a conditional increase or a conditional decrease in the downstream function relative to a basal level of a cgRNA-mediated activity on the target nucleic acid.

In some embodiments, the effector handle region is configured to interact and form a complex with an effector protein selected from the group consisting of Cas9, dCas9, C2C2, Cas13d, any protein fusions or derivatives thereof, any RNA-guided CRISPR effector protein or protein complex, or any protein from a similar pathway.

In some embodiments, the cgRNA comprises one or more chemical modifications that alter one or more of degradation properties, affinity, biological activity, and delivery properties of the cgRNA.

In some embodiments, the one or more chemical modifications is selected from the group consisting of arabino nucleic acids (ANA), locked nucleic acids (LNA), peptide nucleic acids (PNA), phosphoroamidate DNA analogues, phosphorodiamidate morpholino oligomers (PMO), cyclohexene nucleic acids (CeNA), tricycloDNA (tcDNA), bridged nucleic acids (BNA), phosphorothioate modification, 2′-fluoro (2′-F) modification, 2′-fluoroarabino (2′-FANA) modification, 2′O-Methyl (2′O-Me) modification, and 2′O-(2-methoxyethyl) (2′O-MOE) modification.

In some embodiments, a sequence of the cgRNA may be a subsequence of a longer RNA, DNA, or another polymer capable of base-pairing.

In some embodiments, one or more secondary structures formed by the domains of the cgRNA and/or cgRNA-input target complex that are complementary to each other may contain one or more of mismatches, loops, multiloops or bulges due to base-pairing interactions within or between any of the cgRNA domains and input target domains.

In some embodiments, the cgRNA may be expressed in the cells, living organisms or artificial settings in which it interacts with effector, input, and/or target, or may be synthesized exogenously and introduced.

In some embodiments, the cgRNA may conditionally perform a downstream function on a target nucleic acid in one or more of living organisms, ecosystems, tissue extracts, cell lysates, or artificial systems of reconstituted biological components.

In some embodiments, a sequence of input target may be fully constrained, partially constrained, or fully unconstrained by the sequence of target nucleic acid.

In some embodiments, a sequence of input target may be a subsequence of a longer RNA, DNA, or another polymer capable of base-pairing.

In some embodiments, the target nucleic acid may be RNA, DNA, or another polymer capable of base-pairing, coding or non-coding, endogenous or exogenous.

In some embodiments, the RNA-guided effector is selected from the group consisting of Cas9, dCas9, C2C2, Cas13d, protein fusions or derivatives thereof, RNA-guided effector protein or protein complex, any protein from a similar pathway, or any protein the mediates a downstream function on a target nucleic acid in complex with a cgRNA with an active status.

In some embodiments, a cgRNA is provided that comprises a target binding region, configured to bind to a target nucleic acid and an effector handle region. The cgRNA is configured to interact and form a complex with an RNA-guided effector, and the cgRNA is configured to conditionally perform a downstream function on the target nucleic acid in an absence of an input target, the cgRNA comprises from 5′ to 3′ the target binding region comprising a domain a, the effector handle, and an optional terminator region, wherein the cgRNA is configured to be active. In addition, the cgRNA is inactivated by the binding of a domain a* of an input target and the domain a of the target binding region to each other.

In some embodiments, the downstream function is selected from the group consisting of activating an expression of the target nucleic acid, silencing an expression of the target nucleic acid, editing the target nucleic acid, and binding the target nucleic acid.

In some embodiments, the effector handle region is configured to interact and form a complex with an effector protein selected from the group consisting of Cas9, dCas9, C2C2, Cas13d, any protein fusions or derivatives thereof, any RNA-guided CRISPR effector protein or protein complex, any protein from a similar pathway, or any protein the mediates a downstream function on a target nucleic acid in complex with a cgRNA with an active status. In some embodiments, the cgRNA comprises one or more chemical modifications that alter one or more of degradation properties, affinity, biological activity, and delivery properties of the cgRNA. In some embodiments, the one or more chemical modifications is selected from the group consisting of arabino nucleic acids (ANA), locked nucleic acids (LNA), peptide nucleic acids (PNA), phosphoroamidate DNA analogues, phosphorodiamidate morpholino oligomers (PMO), cyclohexene nucleic acids (CeNA), tricycloDNA (tcDNA), bridged nucleic acids (BNA), phosphorothioate modification, 2′-fluoro (2′-F) modification, 2′-fluoroarabino (2′-FANA) modification, 2′O-Methyl (2′O-Me) modification, and 2′O-(2-methoxyethyl) (2′O-MOE) modification. In some embodiments, a sequence of the cgRNA may be a subsequence of a longer RNA, DNA, or another polymer capable of base-pairing. In some embodiments, one or more secondary structures formed by the domains of the cgRNA and/or cgRNA-input target complex that are complementary to each other may contain one or more of mismatches, loops, multiloops or bulges due to base-pairing interactions within or between any of the cgRNA domains and input target domains.

In some embodiments, the cgRNA may be expressed in the cells, living organisms or artificial settings in which it interacts with effector, input, and/or target, or may be synthesized exogenously and introduced. In some embodiments, the cgRNA may conditionally perform a downstream function on a target nucleic acid in one or more of living organisms, ecosystems, tissue extracts, cell lysates, or artificial systems of reconstituted biological components. In some embodiments, a sequence of input target may be fully constrained, partially constrained, or fully unconstrained by the sequence of target nucleic acid. In some embodiments, a sequence of input target may be a subsequence of a longer RNA, DNA, or another polymer capable of base-pairing. In some embodiments, the target nucleic acid may be RNA, DNA, or another polymer capable of base-pairing, coding or non-coding, endogenous or exogenous. In some embodiments, the RNA-guided effector is selected from the group consisting of Cas9, dCas9, C2C2, Cas13d, protein fusions or derivatives thereof, RNA-guided CRISPR effector protein or protein complex, or any protein from a similar pathway.

### BRIEF DESCRIPTION OF THE DRAWINGS

FIG. 1A-FIG. 1C show schematics of the function of the canonical, unconditional gRNA.

FIG. 1A shows an embodiment of a catalytically active RNA-guided effector.

FIG. 1B shows an embodiment of catalytically dead RNA-guided effector.

FIG. 1C shows an embodiment of a RNA-guided effector fusion protein.

FIG. 2 shows an embodiment of molecular logic of RNA-guided effectors with target nucleic acid Y and input target X.

FIG. 3 shows a schematic of an embodiment of a constitutively inactive cgRNA conditionally activated by input target (Toehold Switch: Mechanism 1).

FIG. 4 shows a schematic of an embodiment of a constitutively inactive cgRNA conditionally activated by an input target (Mechanism 2).

FIG. 5 shows a schematic of an embodiment of a constitutively inactive cgRNA conditionally activated by input target (Mechanism 3).

FIG. 6 shows a schematic of an embodiment of a constitutively inactive cgRNA conditionally activated by an input target (Mechanism 4).

FIG. 7 shows a schematic of an embodiment of a constitutively inactive cgRNA conditionally activated by an input target (Mechanism 5).

FIG. 8 shows a schematic of an embodiment of a constitutively active cgRNA conditionally inactivated by an input target (Mechanism 6).

FIG. 9 shows a schematic of an embodiment of a constitutively active cgRNA conditionally inactivated by an input target (Mechanism 7).

FIG. 10 shows a schematic of an embodiment of a constitutively active cgRNA conditionally inactivated by an input target (Splinted Switch A: Mechanism 8).

FIG. 11 shows a schematic of an embodiment of a constitutively active cgRNA conditionally inactivated by an input target (Splinted Switch B: Mechanism 9).

FIG. 12 shows a schematic of an embodiment of a constitutively active cgRNA conditionally inactivated by an input target (Terminator Switch: Mechanism 10).

FIG. 13 shows a schematic of an embodiment of a constitutively active cgRNA conditionally inactivated by an input target (Mechanism 11).

FIG. 14A and FIG. 14B show an embodiment of in vitro cleavage of specific target nucleic acids by specific constitutively active cgRNAs conditionally inactivated by specific input targets (Constitutively Active Splinted Switch A: Mechanism 8).

FIG. 15 shows an embodiment of silencing of expression of specific target nucleic acids by specific constitutively active cgRNAs, conditionally inactivated by specific input targets in E. coli expressing RNA-guided effector dCas9 (Constitutively Active Splinted Switch B: Mechanism 9).

FIG. 16A and FIG. 16B show an embodiment of silencing of expression of specific target nucleic acids by specific constitutively active cgRNAs, conditionally inactivated by specific input targets in E. coli expressing RNA-guided effector dCas9 (Constitutively Active Terminator Switch: Mechanism 10).

FIG. 17A and FIG. 17B show an embodiment of silencing of expression of specific target nucleic acids by specific constitutively inactive cgRNAs, conditionally activated by specific input targets in E. coli expressing RNA-guided effector dCas9 (Constitutively Inactive Toehold Switch: Mechanism 1).

FIG. 18A shows a schematic of an embodiment of a constitutively inactive cgRNA that conditionally performs a downstream function on a target nucleic acid in the presence of an input target and an RNA-guided effector.

FIG. 18B shows a schematic of an embodiment of a constitutively active cgRNA that conditionally performs a downstream function on a target nucleic acid in the absence of an input target and the presence of an RNA-guided effector.

FIGS. 19A and 19B depict nucleotide sequences used in FIGS. 14A and 14B respectively.

FIG. 20 depicts nucleotide sequences used in FIG. 15.

FIG. 21A depicts nucleotide sequences used in FIG. 16A.

FIG. 21B depicts nucleotide sequences used in FIG. 16B.

FIG. 22A depicts nucleotide sequences used in FIG. 17A.

FIG. 22B depicts nucleotide sequences used in FIG. 17B.

### DETAILED DESCRIPTION

The CRISPR/Cas system exists in nature as a prokaryotic immune system, enabling nucleic acid sequence-specific acquired immunity to foreign genetic elements (Barrangou et al. 2007; Horvath and Barrangou 2010). Recent developments in the engineering and implementation of RNA-guided CRISPR effectors have allowed for the high-fidelity sequence-specific interaction of RNA-guided effectors with target nucleic acids in a variety of organisms and settings for a number of applications (Sander and Joung 2014). Previous demonstrations of the implementation of RNA-guided CRISPR effectors include genome editing using the active endonuclease (Cong et al. 2013; Mali et al. 2013), gene regulation and knockdown via inhibition of transcriptional elongation (Qi et al. 2013) or transcriptional activation/repression using regulatory elements fused to the RNA-guided effector (Gilbert et al. 2013), RNA cleavage and editing (Abudayyeh et al. 2016), localization of RNA-guided effector-associated fluorophores for the visualization of genomic loci (Chen et al. 2013), and propagation of engineered genetic traits to whole populations of organisms via gene drives (DiCarlo et al. 2015).

The specificity of the interaction between the guide RNA/CRISPR effector complex and the target nucleic acid is dependent on the sequence of the target-binding region in the guide RNA (gRNA) and on the presence of a protospacer adjacent motif (PAM) (Jinek et al. 2012) or protospacer flanking site (PFS) (Abudayyeh et al. 2016)—a short CRISPR effector-specific sequence adjacent to the target sequence in the target nucleic acid. The gRNA may be a single strand or complex of strands, with a programmable target binding region and an effector handle that has a structure and sequence specific to a particular RNA-guided CRISPR effector (Jinek et al. 2012).

The conceptual power of RNA-guided CRISPR effectors derives from their programmability. A new target nucleic acid can be addressed by changing the sequence of the target-binding region of the gRNA. However, the fact that the gRNA is constitutively active is a significant limitation, making it difficult to control the location and time where the interaction between the gRNA/CRISPR effector complex and the target nucleic acid occurs.

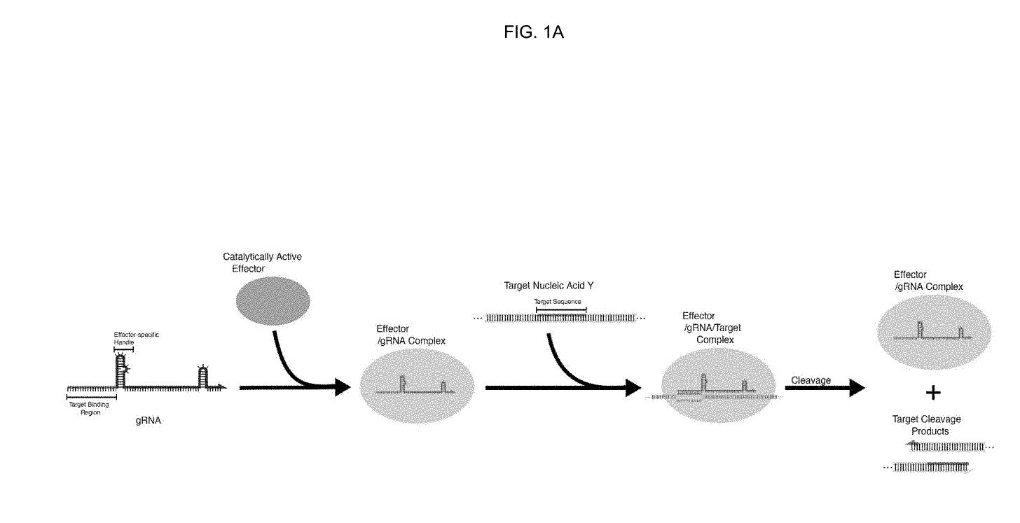

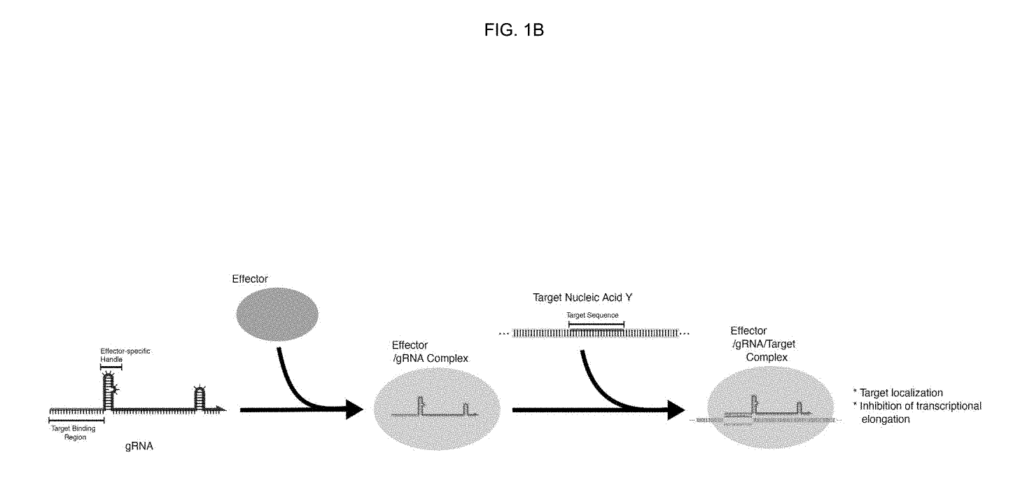

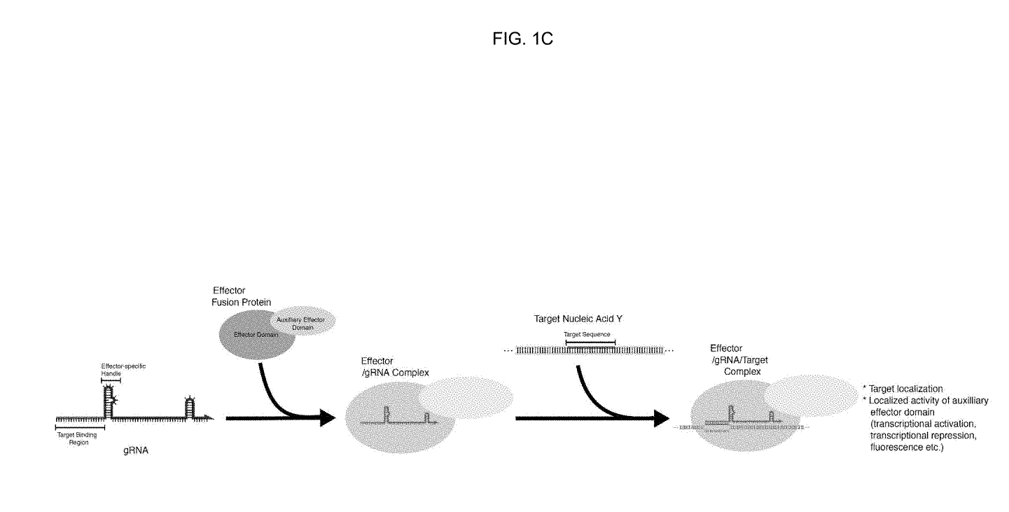

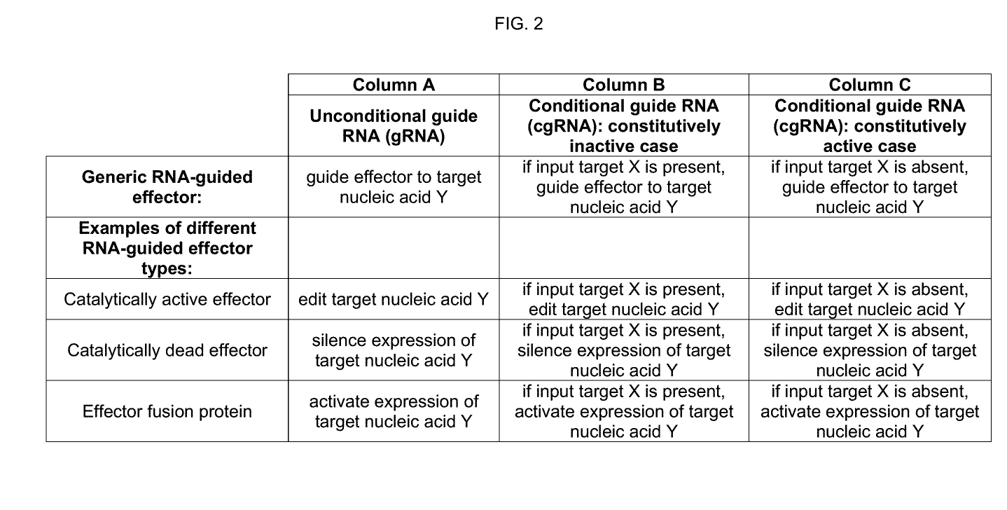

In effect, a gRNA implements an unconditional molecular logic, i.e., guide the RNA-guided CRISPR effector to target nucleic acid Y (FIG. 1A-FIG. 1C and FIG. 2, Column A). FIG. 1A shows a schematic of a catalytically active RNA-guided effector. The catalytically active RNA-guided effector binds the effector handle of the gRNA. The target binding region of the gRNA allows sequence-specific interaction of the effector/gRNA complex and the complementary target sequence of target nucleic acid Y (with appropriate protospacer adjacent motif), forming an effector/gRNA/target complex and mediating site-specific cleavage or editing of the target nucleic acid. FIG. 1B shows a catalytically dead RNA-guided effector. The catalytically dead RNA-guided effector binds the effector handle of the gRNA. The target binding region of the gRNA mediates site-specific localization of the effector/gRNA complex on the target nucleic acid, which may be utilized for inhibition of transcriptional elongation, resulting in silencing of expression of the target nucleic acid. FIG. 1C shows an RNA-guided effector fusion protein. The gRNA is bound by the RNA-guided effector fusion protein, with the canonical RNA-guided effector domain and an auxiliary fused protein domain. The target binding region of the gRNA mediates site-specific localization of the RNA-guided effector/gRNA complex on the target nucleic acid, which results in localized activity of the auxiliary fused protein domain (transcriptional activation, transcriptional inhibition, fluorescence, etc.). FIG. 2, Column A shows a molecular logic of interaction for traditional unconditional guide RNAs (gRNAs). Thus, a spatiotemporal control over the interaction between the CRISPR effector and target nucleic acid is lacking with traditional unconditional gRNAs.

### Conditional Guide RNAs

In order to allow for spatiotemporal control over the interaction between the RNA-guided effector and target nucleic acid, in some embodiments, described herein are conditional guide RNAs (cgRNAs) that perform shape and sequence transduction to implement a conditional molecular logic. In some embodiments, the conditional molecular logic comprises a constitutively inactive cgRNA, which guides an RNA-guided effector to a target nucleic acid Y in the presence of an input target X (FIG. 2, Column B, a constitutively inactive cgRNA that is conditionally activated by an input target X). In some embodiments, the conditional molecular logic comprises a constitutively active cgRNA, which guides an RNA-guided effector to a target nucleic acid Y in the absence of an input target X (FIG. 2, Column C, a constitutively active cgRNA that is conditionally inactivated by an input target X).

In some embodiments, a conditional guide RNA (cgRNA) is described, the cgRNA is configured to change its activity status depending on a presence or an absence of an input target. In some embodiments, the cgRNA forms a complex with an RNA-guided effector and is configured to bind to a specific target nucleic acid.

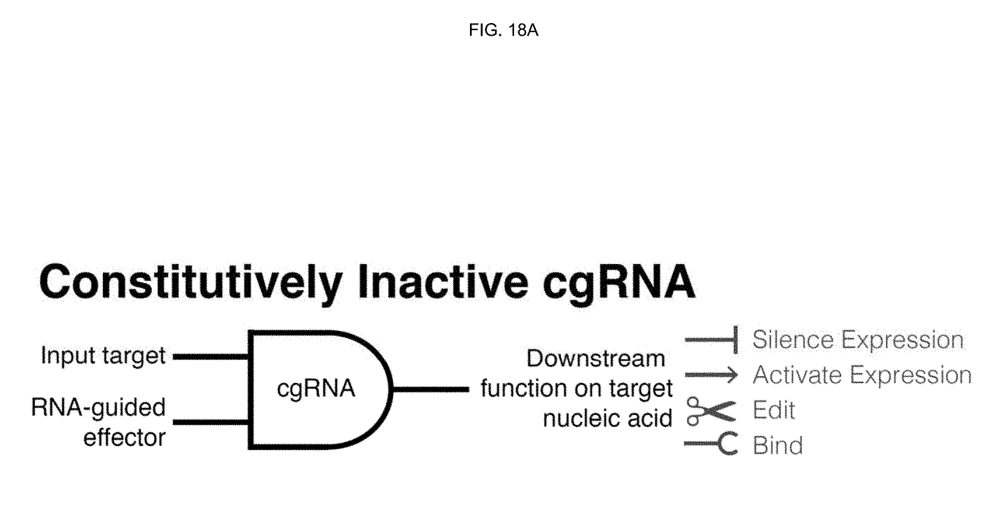

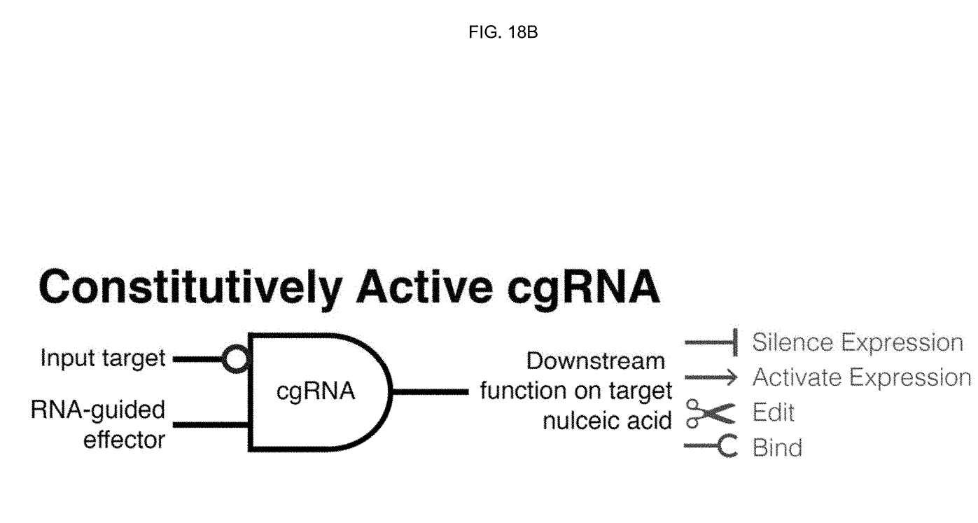

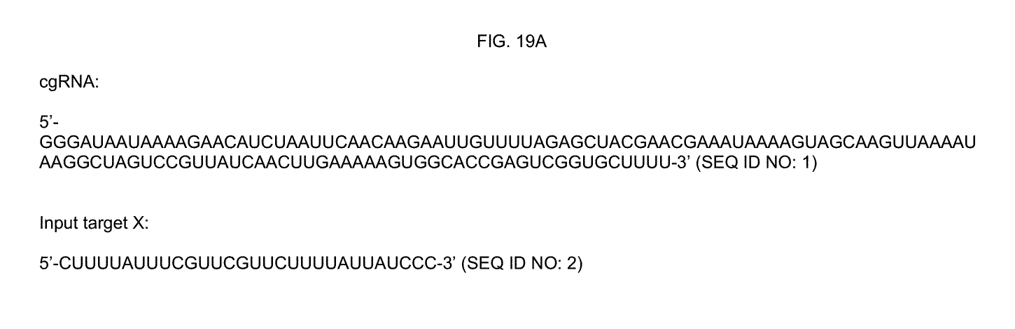

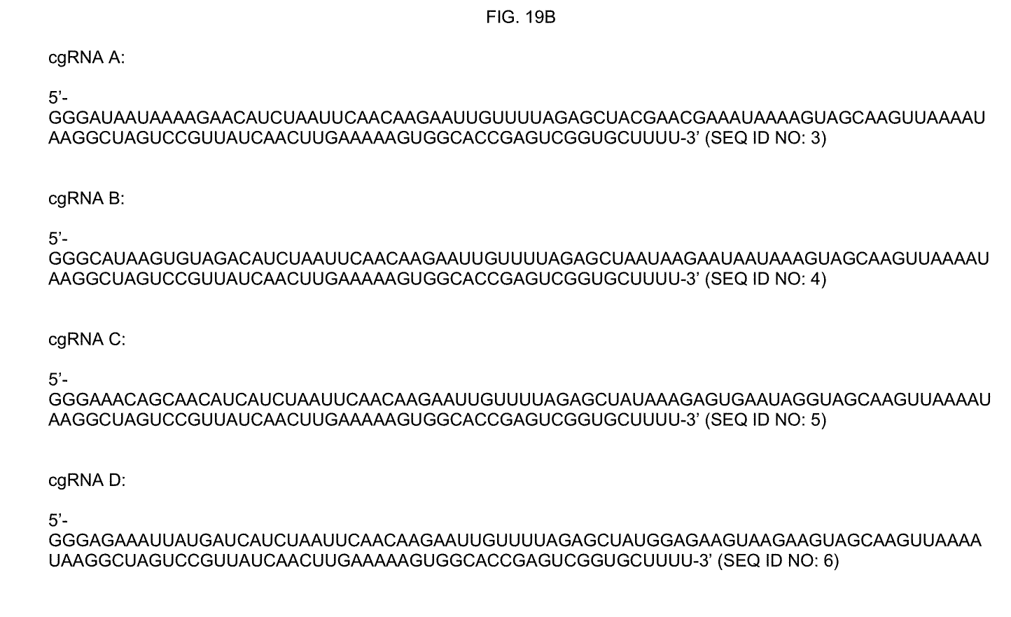

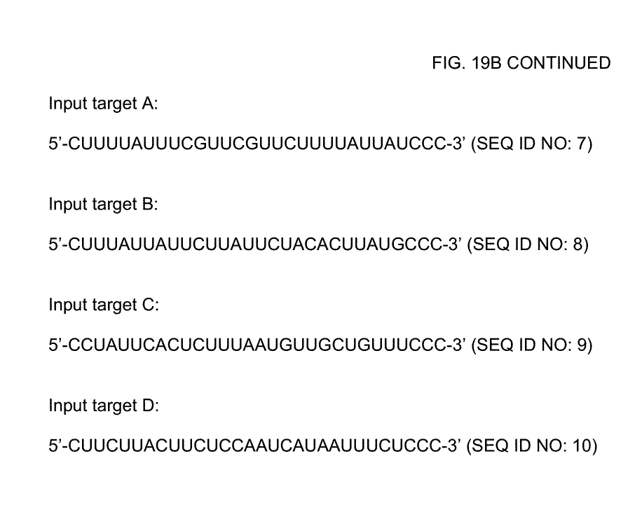

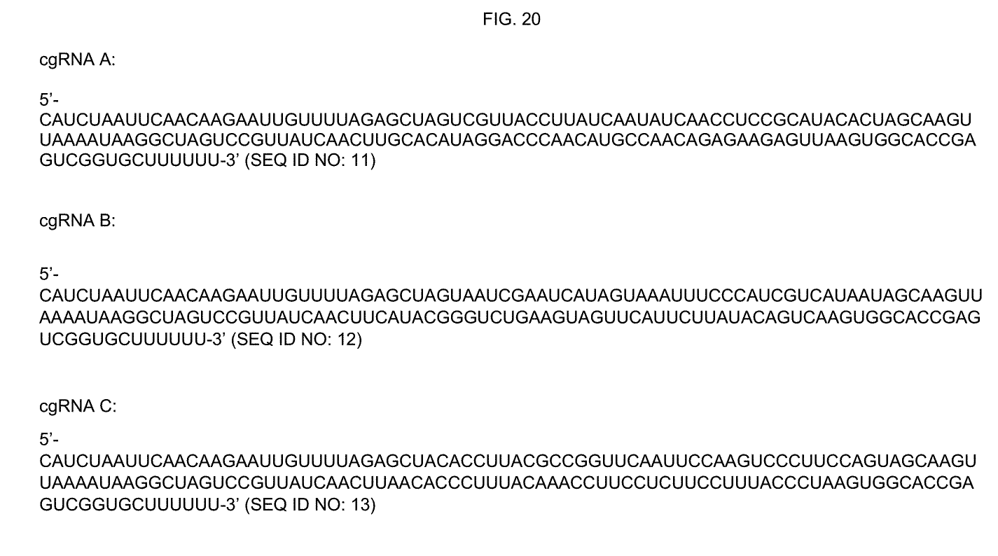

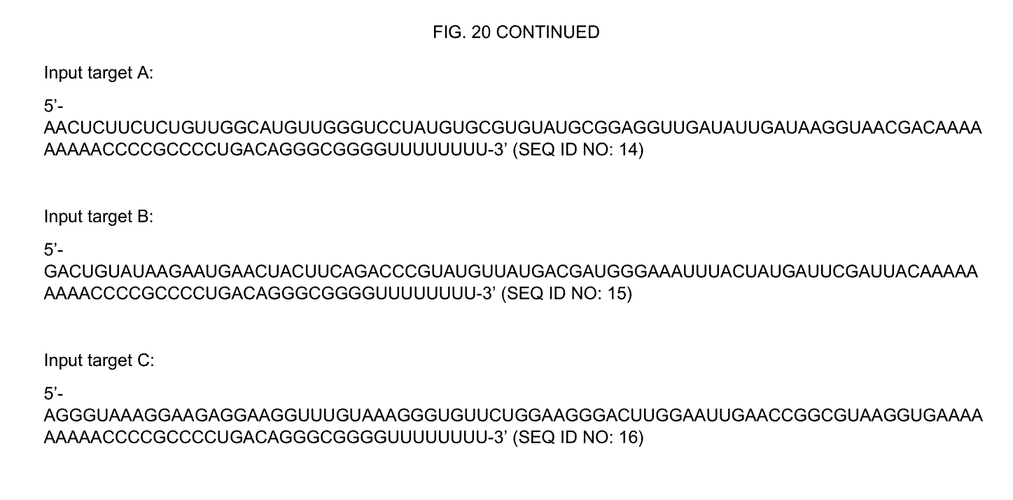

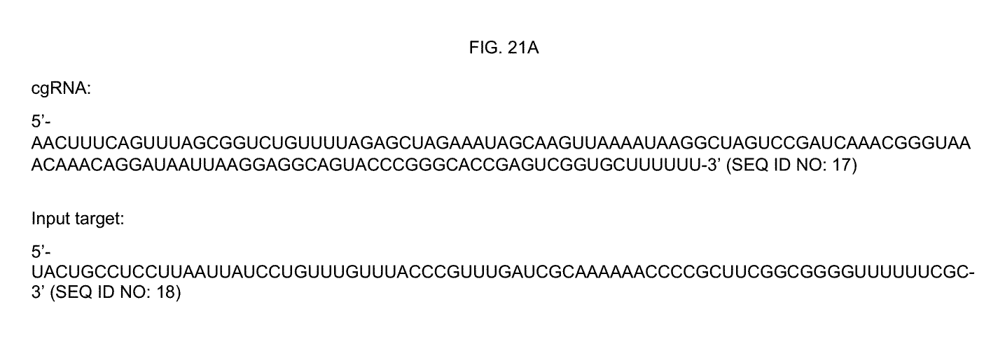

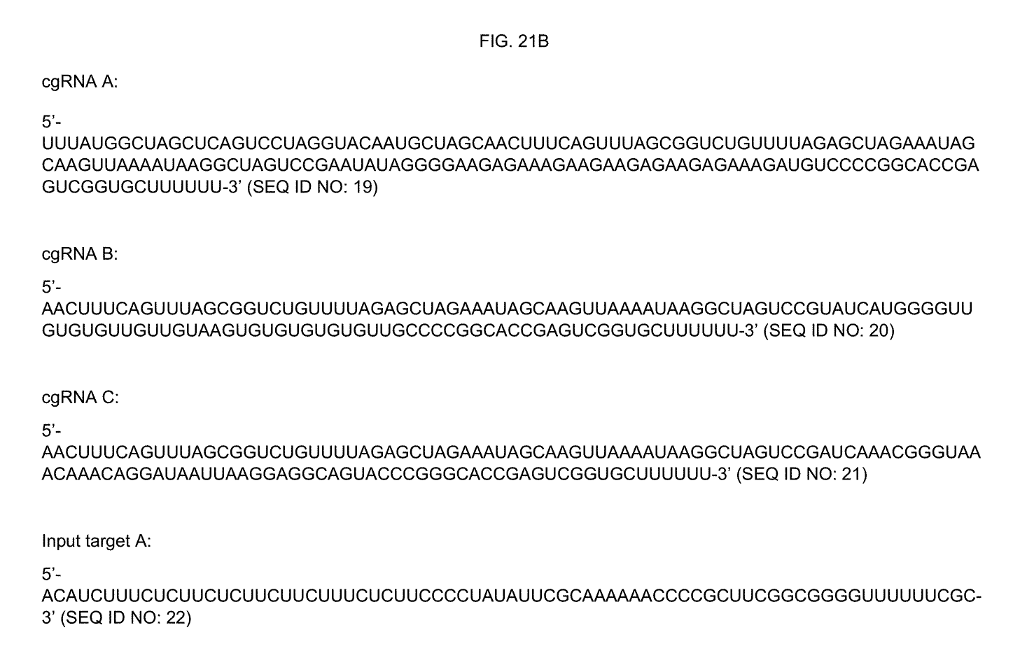

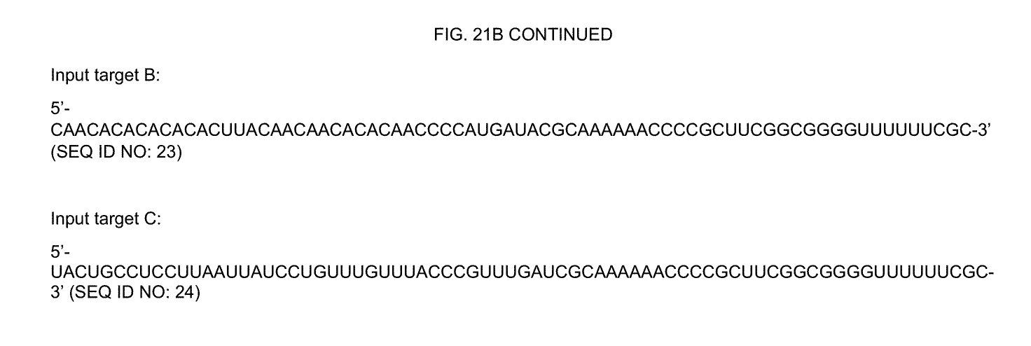

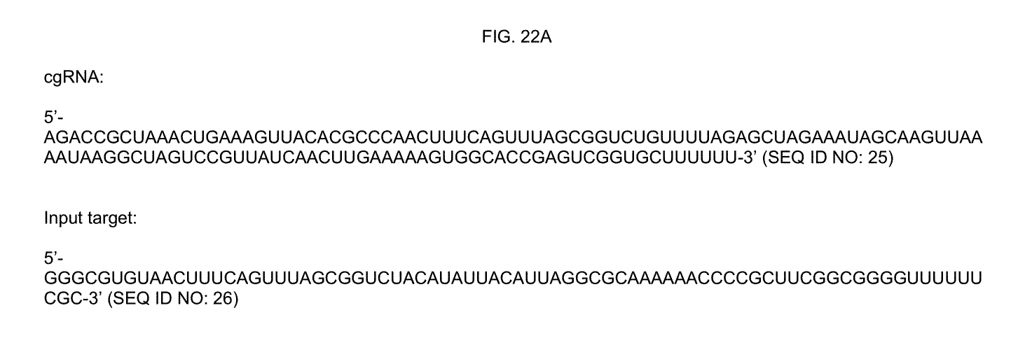

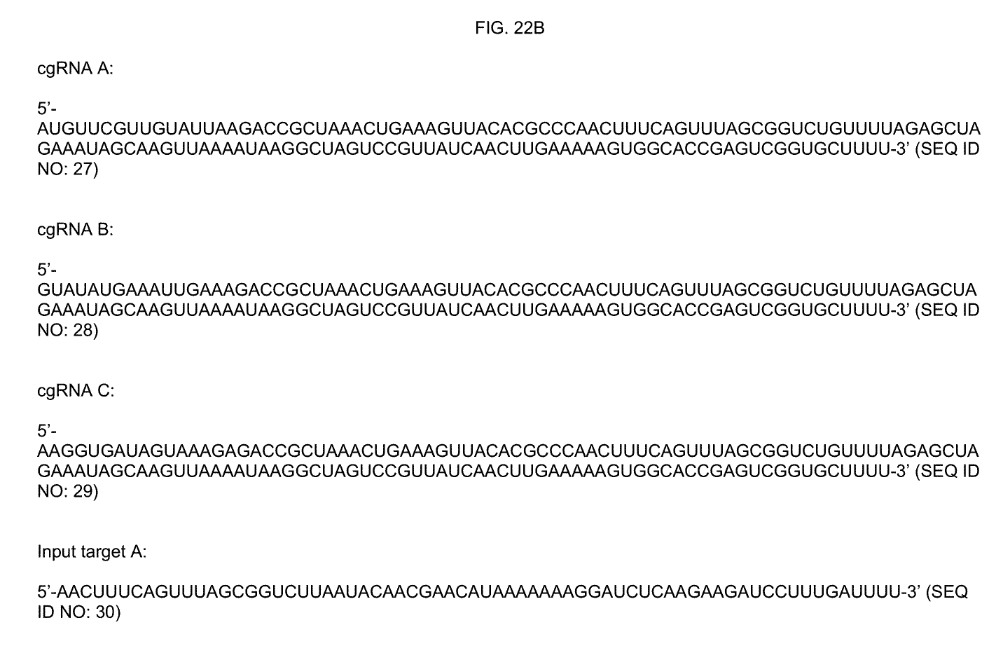

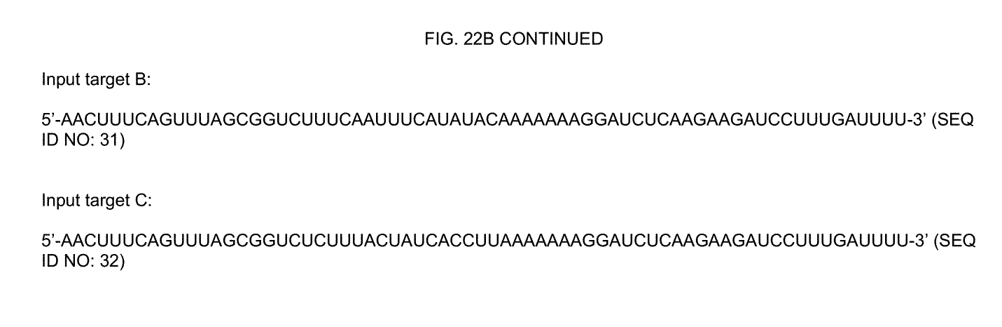

In some embodiments, a cgRNA comprises an input target binding region, a target binding region, and an effector handle. In some embodiments, the input target binding region is configured to bind to an input target (X). In some embodiments, the target binding region is configured to bind to a target nucleic acid (Y). In some embodiments, the cgRNA is configured to interact and form a complex with an RNA-guided effector (FIG. 18A and FIG. 18B). In some embodiments, configured to conditionally perform a downstream function on the target nucleic acid (Y) in a presence of the input target (X) and an RNA-guided effector (FIG. 18A and FIG. 18B). As used herein, “conditionally perform a downstream function” refers to mediation and/or facilitation of a downstream function.

In some embodiments, a cgRNA comprises an input target binding region, a target binding region, and an effector handle. In some embodiments, the input target binding region is configured to bind to an input target (X). In some embodiments, the target binding region is configured to bind to a target nucleic acid (Y). In some embodiments, the cgRNA is configured to interact and form a complex with an RNA-guided effector. In some embodiments, the cgRNA is configured to interact and form a complex with an RNA-guided effector and configured to conditionally perform a downstream function on the target nucleic acid (Y) in an absence of the input target (X).

As used herein, “constitutively inactive cgRNA” (e.g., FIG. 18A) denotes a cgRNA configured to perform a downstream function on a target nucleic acid in the presence of an input target and the presence of an RNA-guided effector. The constitutively inactive cgRNA is conditionally activated by the presence of the input target.

As used herein, “constitutively active cgRNA” (e.g., FIG. 18B) denotes a cgRNA configured to perform a downstream function on a target nucleic acid in the absence of an input target and the presence of an RNA-guided effector. The constitutively active cgRNA is conditionally inactivated by the presence of the input target.

As used herein, “conditionally activated” denotes an increase in the activity status of the cgRNA resulting from the presence of the input target, corresponding to an increased ability to perform a downstream function on a target nucleic acid.

As used herein, “conditionally inactivated” denotes a decrease in the activity status of the cgRNA resulting from the presence of the input target, corresponding to a decreased ability to perform a downstream function on a target nucleic acid.

As used herein, “input target binding region” denotes the region of the cgRNA, comprising one or more sequence domains, that have full or partial sequence complementarity to the input target.

As used herein, “target binding region” denotes the region of the gRNA or cgRNA, comprising one or more sequence domains, that has full or partial sequence complementarity to the target nucleic acid, mediating sequence-specific interaction of the cgRNA/RNA-guided effector complex with the target nucleic acid.

As used herein, “effector handle” denotes the region of the gRNA or cgRNA with effector-specific structure and sequence that binds to the RNA-guided effector.

As used herein, “RNA-guided effector” denotes a protein or protein complex that binds to the effector handle and mediates a downstream function on a target nucleic acid when guided to the target nucleic acid by a gRNA, or by a constitutively inactive cgRNA conditionally activated in the presence of an input target, or by a constitutively active cgRNA in the absence of an input target that would otherwise conditionally inactivate the cgRNA.

As used herein, “terminator region” denotes a region within the gRNA or cgRNA that is 3′ of the effector handle that at least serves as a full or partial transcriptional terminator in the natural host of the RNA-guided effector.

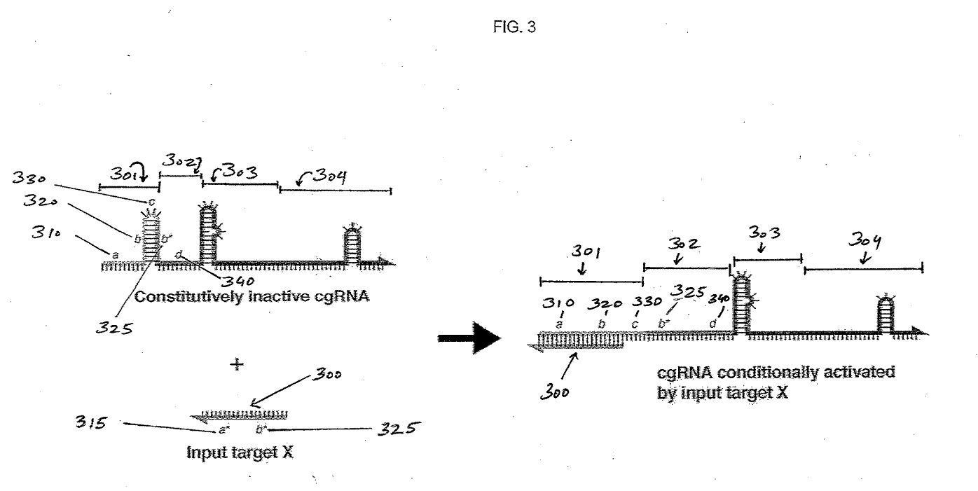

In some embodiments, a cgRNA is configured to conditionally perform a downstream function on the target nucleic acid (Y) by Mechanism 1 (FIG. 3). In some embodiments, the cgRNA comprises from 5′ to 3′ the 5′ extension region (301) comprising a domain a (310), a domain b (320), and a domain c (330), the target binding region (302) comprising a domain b* (325), and a domain d (340), the effector handle region (303), and an optional terminator region (304), wherein the domain b (320) of the 5′ extension region (301) and the domain b* (325) of the target binding region (302) are complementary to each other, and wherein the cgRNA is configured to be inactive by the binding of the domain b (320) of the 5′ extension region (301) and the domain b* (325) of the target binding region (302) to each other (FIG. 3). Brackets in this and other drawings designate the denoted item referenced in the figure. Additional sequences can be inserted between each bracketed section or other section displayed in the figure as desired, in some embodiments. The figures are representative only, and do not limit or define the figures.

In some embodiments, a cgRNA configured to conditionally perform a downstream function on the target nucleic acid (Y) by Mechanism 1 comprises from 5′ to 3′ the 5′ extension region (301) comprising a domain a (310) 0-200 nucleotides in length, a domain b (320) 4-200 nucleotides in length, and a domain c (330) 1-200 nucleotides in length, and the target binding region (302) comprising a domain b* (325) 4-200 nucleotides in length, and a domain d (340) 0-20 nucleotides in length. In some embodiments, the cgRNA comprises from 5′ to 3′ the 5′ extension region (301) comprising a domain a (310) 15 or 5-30 nucleotides in length, a domain b (320) 20 or 5-40 nucleotides in length, and a domain c (330) 4 or 1-30 nucleotides in length, and the target binding region (302) comprising a domain b* (325) 20 or 5-40 nucleotides in length, and a domain d (340) 0 or 1-8 nucleotides in length. In some embodiments, domain a, domain b, domain c, domain b*, and domain d may be any lengths.

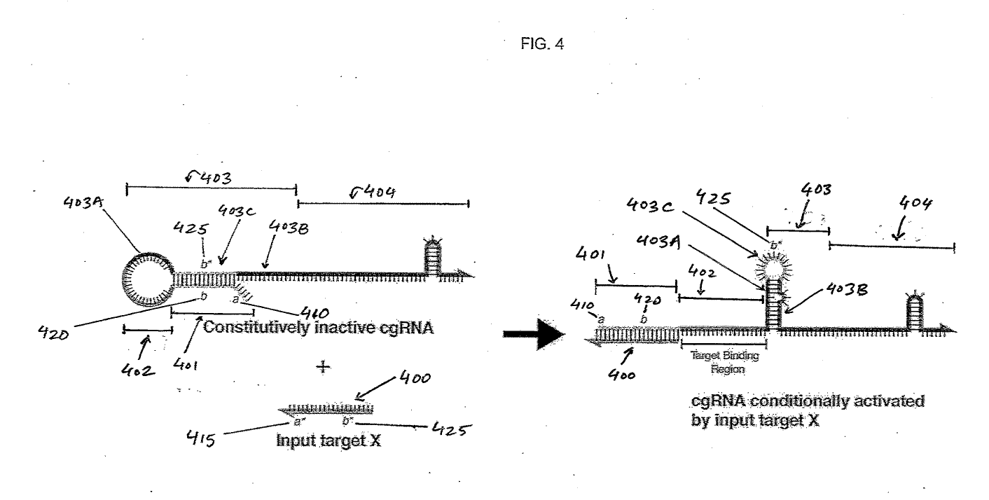

In some embodiments, a cgRNA is configured to conditionally perform a downstream function on the target nucleic acid (Y) by Mechanism 2 (FIG. 4). In some embodiments, the cgRNA comprises from 5′ to 3′ the 5′ extension region (401) comprising a domain a (410), and a domain b (420), the target binding region (402), a first partial sequence (403A) of the effector handle (403), a modified effector handle loop region (403C) comprising a domain b* (425), a second partial sequence (403B) of the effector handle (403), and an optional terminator region (404), wherein the domain b (420) of the 5′ extension region (401) and the domain b* (425) of the modified effector handle loop region (403C) are complementary to each other, and wherein the cgRNA is configured to be inactive by the binding of the domain b (420) of the 5′ extension region (401) and the domain b* (425) of the modified effector handle loop region (403C) to each other (FIG. 4).

In some embodiments, a cgRNA configured to conditionally perform a downstream function on the target nucleic acid (Y) by Mechanism 2 comprises from 5′ to 3′ the 5′ extension region (401) comprising a domain a (410) 0-200 nucleotides in length, and a domain b (420) 3-200 nucleotides in length, and a modified effector handle loop region (403C) comprising a domain b* (425) 3-200 nucleotides in length. In some embodiments, the cgRNA comprises from 5′ to 3′ the 5′ extension region (401) comprising a domain a (410) 15 or 5-30 nucleotides in length, and a domain b (420) 40 or 10-100 nucleotides in length, and a modified effector handle loop region (403C) comprising a domain b* (425) 40 or 10-100 nucleotides in length. In some embodiments, domain a, domain b, and domain b* may be any lengths.

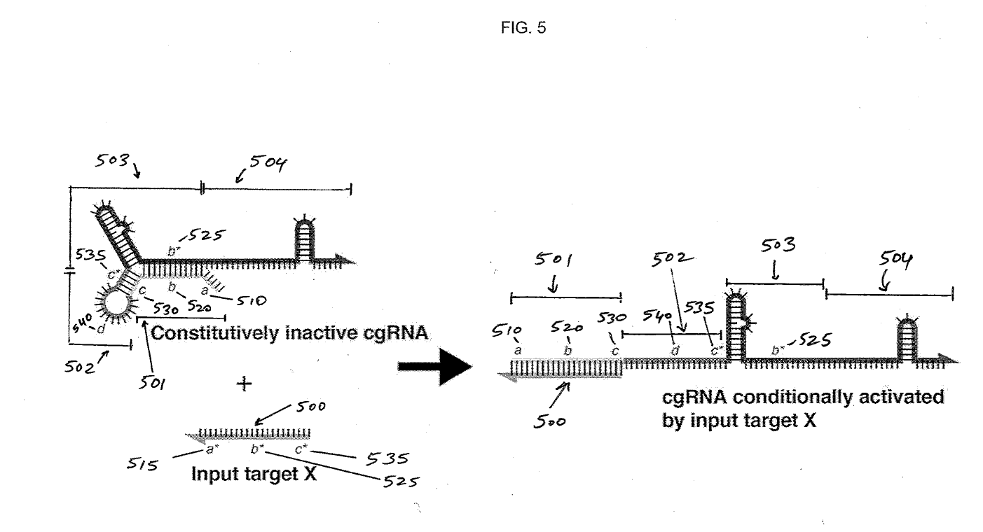

In some embodiments, a cgRNA is configured to conditionally perform a downstream function on the target nucleic acid (Y) by Mechanism 3 (FIG. 5). In some embodiments, the cgRNA comprises from 5′ to 3′ the 5′ extension region (501) comprising a domain a (510), a domain b (520), and a domain c (530), the target binding region (502) comprising a domain d (540), and a domain c* (535), the effector handle (503) comprising a domain b* (525), and an optional terminator (504), wherein the domain b (520) of the 5′ extension region (501) and the domain b* (525) of the effector handle (503) are complementary to each other, wherein the domain c (530) of the 5′ extension region (501) and the domain c* (535) of the target binding region (502) are complementary to each other, and wherein the cgRNA is configured to be inactive by the binding of the domain b (520) of the 5′ extension region (501) and the domain b* (525) of the effector handle (503) to each other, and by the binding of the domain c (530) of the 5′ extension region (501) and the domain c* (535) of the target binding region (502) to each other (FIG. 5).

In some embodiments, a cgRNA configured to conditionally perform a downstream function on the target nucleic acid (Y) by Mechanism 3 comprises from 5′ to 3′ the 5′ extension region (501) comprising a domain a (510) 0-200 nucleotides in length, a domain b (520) 1-150 nucleotides in length, and a domain c (530) 4-200 nucleotides in length, the target binding region (502) comprising a domain d (540) 1-200 nucleotides in length, and a domain c* (535) 4-200 nucleotides in length, and the effector handle (503) comprising a domain b* (525) 1-150 nucleotides in length. In some embodiments, the cgRNA comprises from 5′ to 3′ the 5′ extension region (501) comprising a domain a (510) 15 or 5-30 nucleotides in length, a domain b (520) 10 or 1-40 nucleotides in length, and a domain c (530) 20 or 5-40 nucleotides in length, the target binding region (502) comprising a domain d (540) 4 or 1-30 nucleotides in length, and a domain c* (535) 20 or 5-40 nucleotides in length, and the effector handle (503) comprising a domain b* (525) 10 or 1-40 nucleotides in length. In some embodiments, domain a, domain b, domain c, domain d, domain c*, and domain b* may be any lengths.

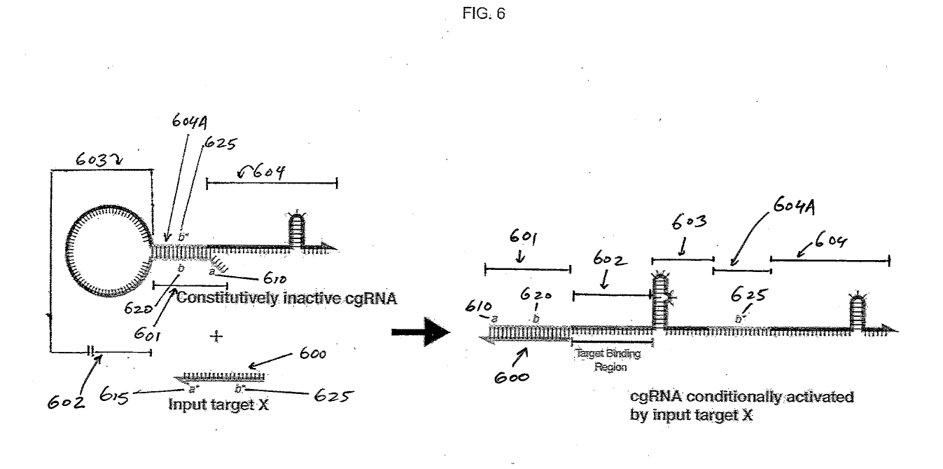

In some embodiments, a cgRNA is configured to conditionally perform a downstream function on the target nucleic acid (Y) by Mechanism 4 (FIG. 6). In some embodiments, the cgRNA comprises from 5′ to 3′ the 5′ extension region (601) comprising a domain a (610), and a domain b (620), the target binding region (602), the effector handle (603), a terminator insert region (604A) comprising domain b* (625), and a terminator region (604), wherein domain b (620) of the 5′ extension region (601) and the domain b* (625) of the terminator insert region (604A) are complementary to each other, and wherein the cgRNA is configured to be inactive by the binding of domain b (620) of the 5′ extension region (601) and the domain b* (625) of the terminator insert region (604A) to each other (FIG. 6).

In some embodiments, a cgRNA configured to conditionally perform a downstream function on the target nucleic acid (Y) by Mechanism 4 comprises from 5′ to 3′ the 5′ extension region (601) comprising a domain a (610) 0-200 nucleotides in length, and a domain b (620) 3-200 nucleotides in length, and a terminator insert region (604A) comprising domain b* (625) 3-200 nucleotides in length. In some embodiments, the cgRNA comprises from 5′ to 3′ the 5′ extension region (601) comprising a domain a (610) 15 or 5-30 nucleotides in length, and a domain b (620) 40 or 10-100 nucleotides in length, and a terminator insert region (604A) comprising domain b* (625) 40 or 10-100 nucleotides in length. In some embodiments, domain a, domain b, and domain b* may be any lengths.

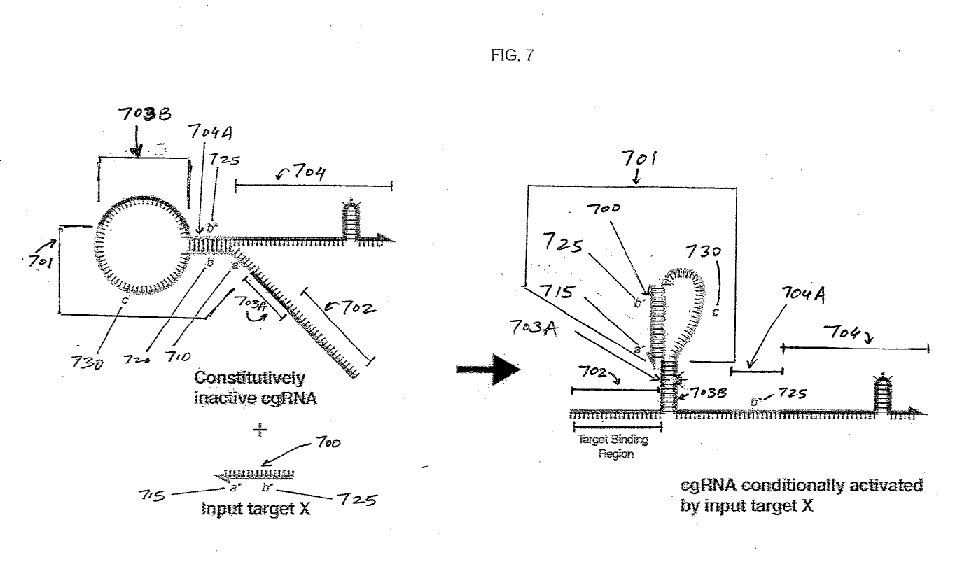

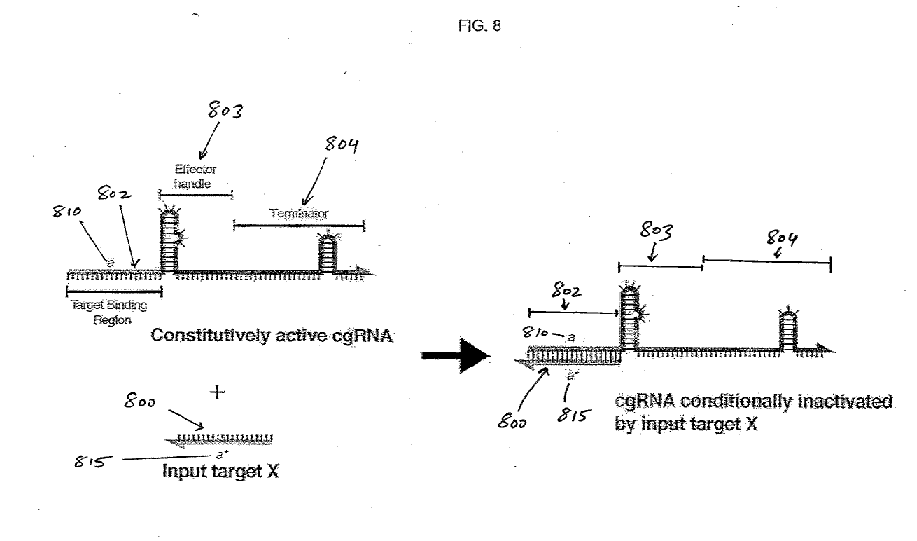

In some embodiments, a cgRNA is configured to conditionally perform a downstream function on the target nucleic acid (Y) by Mechanism 5 (FIG. 7). In some embodiments, the cgRNA comprises from 5′ to 3′ the target binding region (702), a first partial sequence (703A) of the effector handle (703), the modified effector handle loop region (701) comprising a domain a (710), a domain b (720), and a domain c (730), a second partial sequence (703B) of the effector handle (703), a terminator insert region (704A) comprising domain b* (725), and a terminator region (704), wherein the domain b (720) of the modified effector handle loop region (701) and domain b* (725) of the terminator insert region (704A) are complementary to each other, and wherein the cgRNA is configured to be inactive by the binding of the domain b (720) of the modified effector handle loop region (701) and domain b* (725) of the terminator insert region (704A) to each other (FIG. 7).

In some embodiments, a cgRNA configured to conditionally perform a downstream function on the target nucleic acid (Y) by Mechanism 5 comprises from 5′ to 3′ the modified effector handle loop region (701) comprising a domain a (710) 0-200 nucleotides in length, a domain b (720) 3-200 nucleotides in length, and a domain c (730) 0-200 nucleotides in length, and a terminator insert region (704A) comprising domain b* (725) 3-200 nucleotides in length. In some embodiments, the cgRNA comprises from 5′ to 3′ the modified effector handle loop region (701) comprising a domain a (710) 15 or 5-30 nucleotides in length, a domain b (720) 40 or 10-100 nucleotides in length, and a domain c (730) 55 or 15-100 nucleotides in length, and a terminator insert region (704A) comprising domain b* (725) 40 or 10-100 nucleotides in length. In some embodiments, domain a, domain b, domain c, and domain b* may be any lengths.

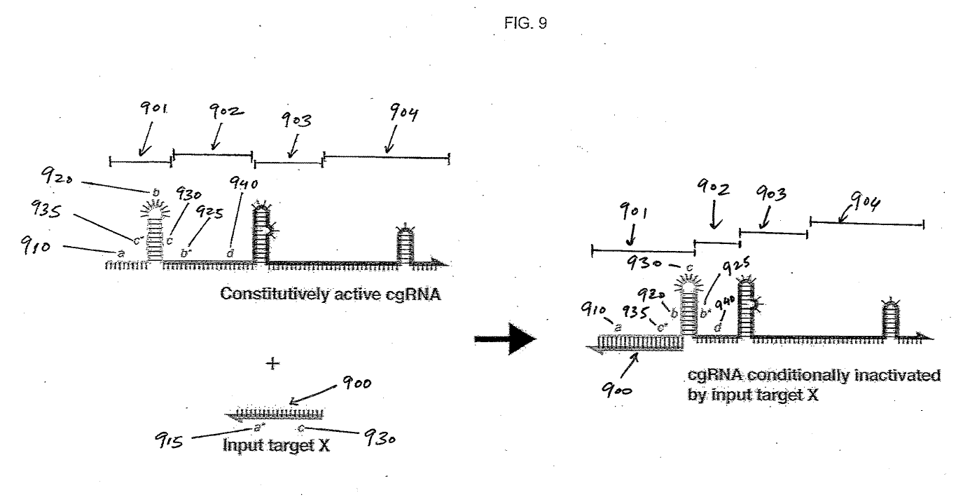

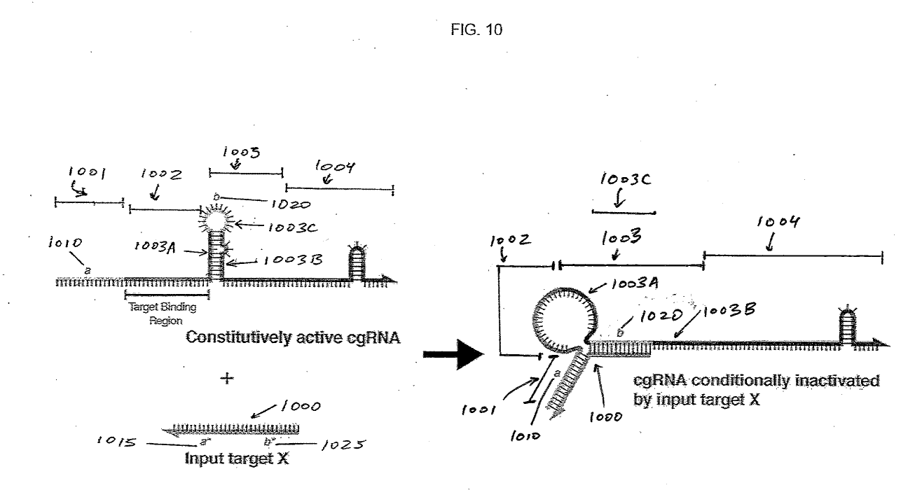

In some embodiments, a cgRNA is configured to conditionally perform a downstream function on the target nucleic acid (Y) by Mechanism 7 (FIG. 9). In some embodiments, the cgRNA comprises from 5′ to 3′ the 5′ extension region (901) comprising a domain a (910), a domain c* (935), a domain b (920), and a domain c (930), the target binding region (902) comprising a domain b* (925), and a domain d (940), the effector handle (903), and an optional terminator region (904), wherein the domain b (920) of the 5′ extension region (901) and the domain b* (925) of the target binding region (902) are complementary to each other, and the domain c (930) and the domain c* (935) of the 5′ extension region (901) are complementary to each other, and wherein the cgRNA is configured to be active by the binding of the domain c (930) and the domain c* (935) of the 5′ extension region (901) to each other (FIG. 9).

In some embodiments, a cgRNA configured to conditionally perform a downstream function on the target nucleic acid (Y) by Mechanism 7 comprises from 5′ to 3′ the 5′ extension region (901) comprising a domain a (910) 0-200 nucleotides in length, a domain c* (935) 3-200 nucleotides in length, a domain b (920) 1-200 nucleotides in length, and a domain c (930) 3-200 nucleotides in length, and the target binding region (902) comprising a domain b* (925) 4-200 nucleotides in length, and a domain d (940) 0-20 nucleotides in length. In some embodiments, the cgRNA comprises from 5′ to 3′ the 5′ extension region (901) comprising a domain a (910) 15 or 5-30 nucleotides in length, a domain c* (935) 20 or 10-100 nucleotides in length, a domain b (920) 4 or 1-30 nucleotides in length, and a domain c (930) 20 or 10-100 nucleotides in length, and the target binding region (902) comprising a domain b* (925) 20 or 5-40 nucleotides in length, and a domain d (940) 0 or 1-8 nucleotides in length. In some embodiments, domain a, domain c*, domain b, domain c, domain b*, and domain d may be any lengths.

In some embodiments, a cgRNA is configured to conditionally perform a downstream function on the target nucleic acid (Y) by Mechanism 8 (FIG. 10). In some embodiments, the cgRNA comprises from 5′ to 3′ the 5′ extension region (1001) comprising a domain a (1010), the target binding region (1002), a first partial sequence (1003A) of the effector handle (1003), a modified effector handle loop region (1003C) comprising a domain b (1020), a second partial sequence (1003B) of the effector handle (1003), and an optional terminator region (1004), wherein the cgRNA is configured to be active (FIG. 10).

In some embodiments, a cgRNA configured to conditionally perform a downstream function on the target nucleic acid (Y) by Mechanism 8 comprises from 5′ to 3′ the 5′ extension region (1001) comprising a domain a (1010) 3-200 nucleotides in length, and a modified effector handle loop region (1003C) comprising a domain b (1020) 3-200 nucleotides in length. In some embodiments, the cgRNA comprises from 5′ to 3′ the 5′ extension region (1001) comprising a domain a (1010) 40 or 10-100 nucleotides in length, and a modified effector handle loop region (1003C) comprising a domain b (1020) 40 or 10-100 nucleotides in length. In some embodiments, domain a and domain b may be any lengths.

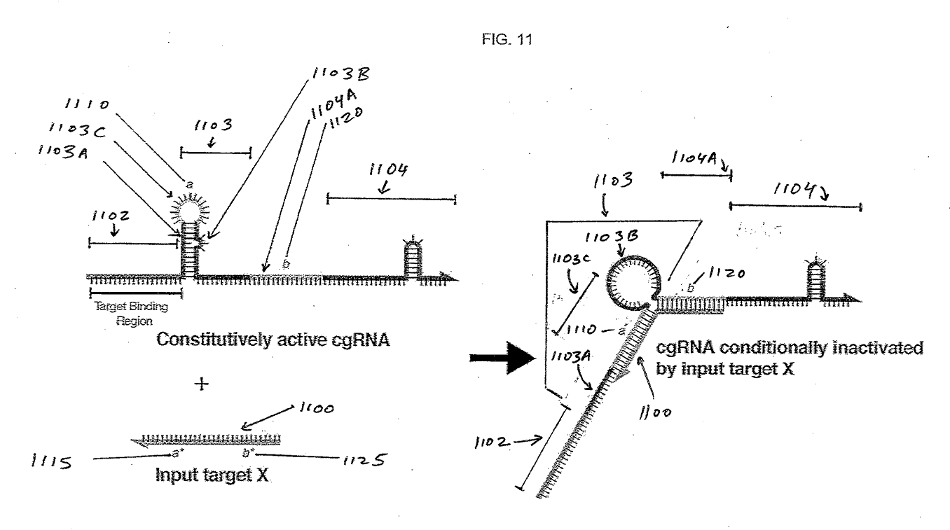

In some embodiments, a cgRNA is configured to conditionally perform a downstream function on the target nucleic acid (Y) by Mechanism 9 (FIG. 11). In some embodiments, the cgRNA comprises from 5′ to 3′ the target binding region (1102), a first partial sequence (1103A) of the effector handle (1103), a modified effector handle loop region (1103C) comprising a domain a (1110), a second partial sequence (1103B) of the effector handle (1103), a terminator insert region (1104A) comprising a domain b (1120), and a terminator region (1104), wherein the cgRNA is configured to be active (FIG. 11).

In some embodiments, a cgRNA configured to conditionally perform a downstream function on the target nucleic acid (Y) by Mechanism 9 comprises from 5′ to 3′ a modified effector handle loop region (1103C) comprising a domain a (1110) 3-200 nucleotides in length, and a terminator insert region (1104A) comprising a domain b (1120) 3-200 nucleotides in length. In some embodiments, the cgRNA comprises from 5′ to 3′ a modified effector handle loop region (1103C) comprising a domain a (1110) 40 or 10-100 nucleotides in length, and a terminator insert region (1104A) comprising a domain b (1120) 40 or 10-100 nucleotides in length. In some embodiments, domain a and domain b may be any lengths.

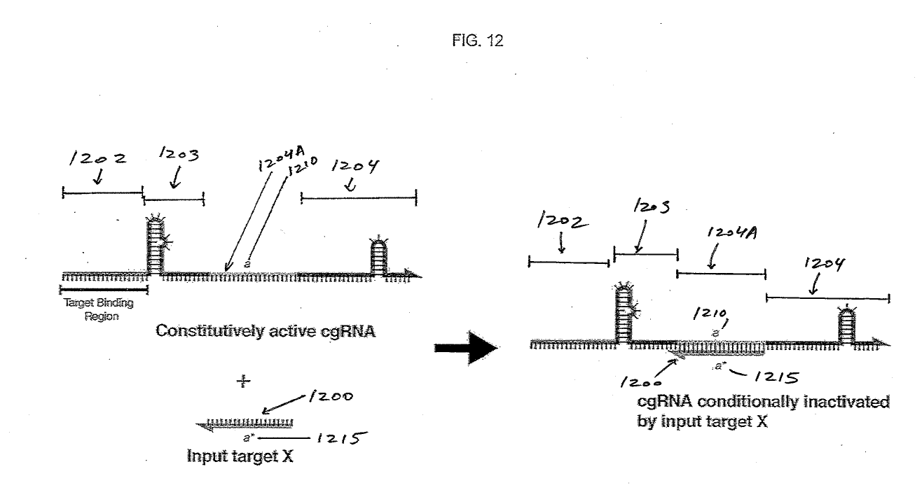

In some embodiments, a cgRNA is configured to conditionally perform a downstream function on the target nucleic acid (Y) by Mechanism 10 (FIG. 12). In some embodiments, the cgRNA comprises from 5′ to 3′ the target binding region (1202), the effector handle (1203), a terminator insert region (1204A) comprising a domain a (1210), and a terminator region (1204), wherein the cgRNA is configured to be active (FIG. 12).

In some embodiments, a cgRNA configured to conditionally perform a downstream function on the target nucleic acid (Y) by Mechanism 10 comprises a terminator insert region (1204A) comprising a domain a (1210) 3-200 nucleotides in length. In some embodiments, the cgRNA comprises a terminator insert region (1204A) comprising a domain a (1210) 40 or 10-100 nucleotides in length. In some embodiments, domain a may be any length.

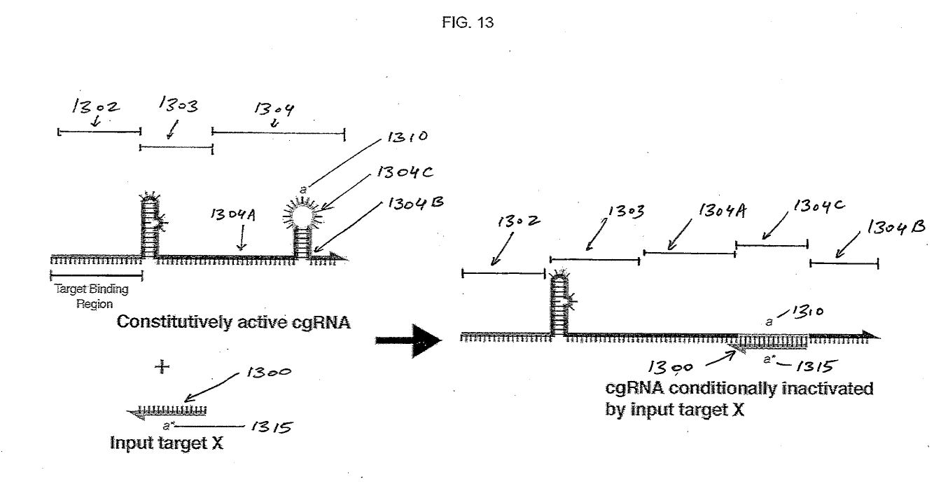

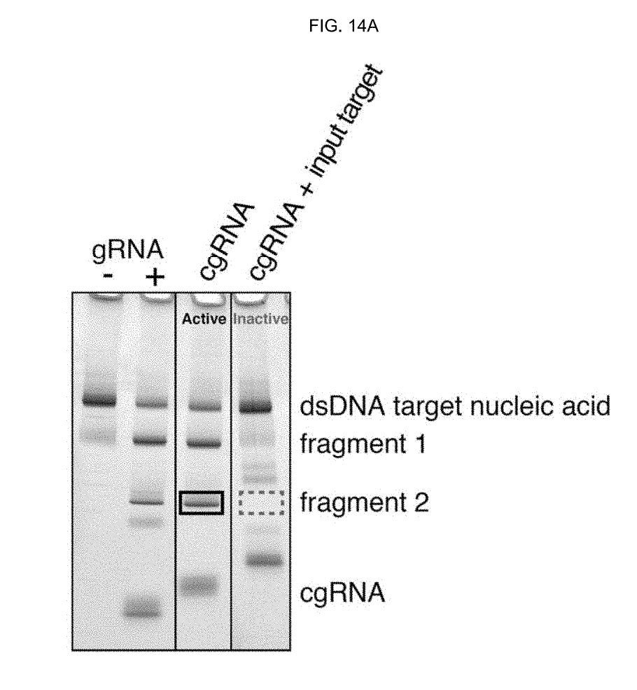

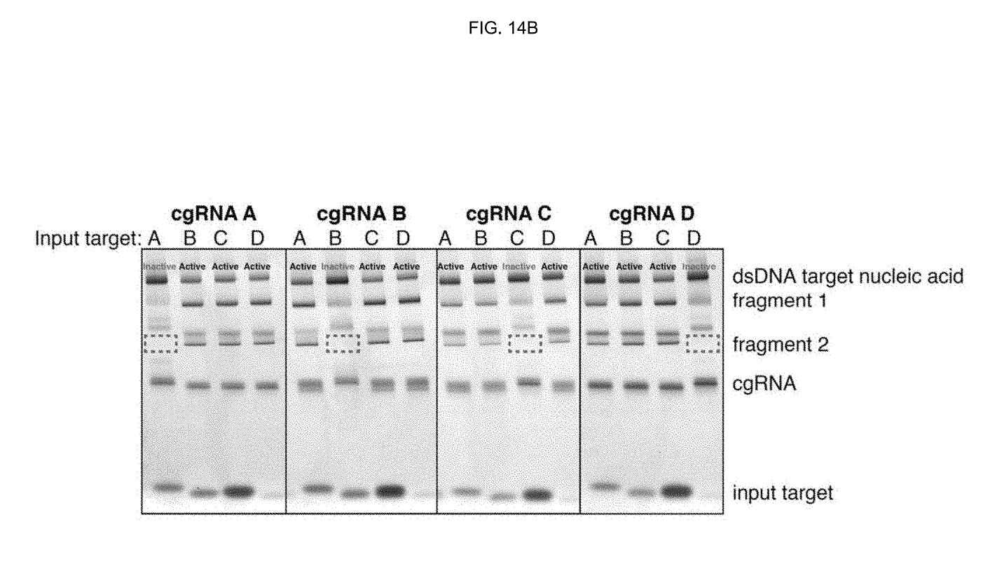

In some embodiments, a cgRNA is configured to conditionally perform a downstream function on the target nucleic acid (Y) by Mechanism 11 (FIG. 13). In some embodiments, the cgRNA comprises from 5′ to 3′ the target binding region (1302), the effector handle (1303), a first partial sequence (1304A) of a terminator region (1304), a modified terminator loop region (1304C) comprising a domain a (1310), and a second partial sequence (1304B) of the terminator region (1304), wherein the cgRNA is configured to be active (FIG. 13).

In some embodiments, a cgRNA configured to conditionally perform a downstream function on the target nucleic acid (Y) by Mechanism 11 comprises a modified terminator loop region (1304C) comprising a domain a (1310) 3-200 nucleotides in length. In some embodiments, the cgRNA comprises a modified terminator loop region (1304C) comprising a domain a (1310) 40 or 10-100 nucleotides in length. In some embodiments, domain a may be any length.

In some embodiments, the cgRNA is configured to conditionally perform a downstream function on the target nucleic acid (Y) by Mechanism 1 such that the input target (300) comprises from 3′ to 5′ a domain a* (315) and the domain b* (325), and wherein the domain a (310) and the domain a* (315) are complementary to each other, and the domain b (320) and the domain b* (325) are complementary to each other, and wherein the cgRNA is configured to be activated by the binding of the domain a (310) of the 5′ extension region (301) and domain a* (315) of the input target (300) to each other and the domain b (320) of the 5′ extension region (301) and the domain b* (325) of the input target (300) to each other (FIG. 3).

In some embodiments, a cgRNA of Mechanism 1 (FIG. 3) can be employed. This can comprise a constitutively inactive cgRNA that is comprised of a modified single stranded gRNA with a 5′ extension abc that forms a hairpin with the domain b* of the target binding region b*d, a canonical effector handle, and an optional terminator region. In the absence of input target X, the b domain of the 5′ extension hybridizes to domain b* of the target binding region, thereby partially sequestering the target binding region and rendering the cgRNA inactive. In the presence of input target X with sequence b*a* complementary to the toehold domain a and stem domain b of the 5′ extension, input target X hybridizes with the ab domains of the 5′ extension, exposing the previously sequestered domain b* of the target binding region. In this way, in the presence of input target X, the target binding region of the constitutively inactive cgRNA is fully accessible, rendering the cgRNA conditionally active. The target binding region may be fully sequestered in the absence of input target X for a zero-length domain d (i.e. where domain b* is the full target binding region). The sequence of input target X is partially constrained by the sequence of target nucleic acid Y due to the common domain b*.

In some embodiments, the cgRNA is configured to conditionally perform a downstream function on the target nucleic acid (Y) by Mechanism 2 such that the input target (400) comprises from 3′ to 5′ a domain a* (415) and the domain b* (425), wherein the domain a (410) and the domain a* (415) are complementary to each other, and wherein the domain b (420) and the domain b* (425) are complementary to each other, and wherein the cgRNA is configured to be activated by the binding of the domain a (410) of the 5′ extension region (401) and domain a* (415) of the input target (400) to each other and domain b of the 5′ extension region (401) and the domain b* (425) of the input target (400) to each other (FIG. 4).

In some embodiments, a cgRNA of Mechanism 2 (FIG. 4) can be employed. This can comprise a constitutively inactive cgRNA that comprises a 5′ extension ab, a target binding region, an effector handle with a modified effector handle loop b*, and an optional terminator region. In the absence of input target X, the 5′ extension domain b forms a hairpin with the modified loop domain b*, disrupting the effector handle and sequestering the target binding region, thus rendering the cgRNA inactive. In the presence of input target X with sequence b*a* complementary to the 5′ extension domains ab, input target X hybridizes to the accessible toehold region a and domain b, exposing the previously sequestered target binding region and effector handle. In this way, in the presence of input target X, the target binding region of the constitutively inactive cgRNA is fully accessible and the effector handle is able to form, rendering the cgRNA active. The sequence of input target X is fully independent of the sequence of target nucleic acid Y. The loop formed in the inactive state may be partially structured, including formation of additional multiloops, interior loops, or bulge loops within the looped region.

In some embodiments, the cgRNA is configured to conditionally perform a downstream function on the target nucleic acid (Y) by Mechanism 3 such that the input target (500) comprises from 3′ to 5′ a domain a* (515), the domain b* (525), and the domain c* (535), wherein the domain a (510) and the domain a* (515) are complementary to each other, and wherein the domain b (520) and the domain b* (525) are complementary to each other, and wherein the domain c (530) and the domain c* (535) are complementary to each other, and wherein the cgRNA is configured to be activated by the binding of the domain a (510) of the 5′ extension region (501) and domain a* (515) of the input target (500) to each other, domain b (520) of the 5′ extension region (501) and the domain b* (525) of the input target (500) to each other, and domain c (530) of the 5′ extension region (501) and the domain c* (535) of the input target (500) to each other (FIG. 5).

In some embodiments, a cgRNA of Mechanism 3 can be employed. This can comprise an arrangement as shown in FIG. 5. This includes a constitutively inactive cgRNA comprising a 5′ extension abc, a target binding region dc*, a canonical effector handle that includes a domain b*, and an optional terminator region. In the absence of input target X, the 5′ extension abc forms a multiloop by hybridizing to the domain b* of the effector handle and the domain c* of the target binding region, sequestering the target binding region and disrupting the secondary structure of the effector handle, thus rendering the cgRNA inactive. In the presence of input target X with sequence c*b*a* complementary to the abc toehold and stem domains of the 5′ extension, input target X hybridizes to the abc domains of the 5′ extension, exposing the previously sequestered target binding region domains dc*. In this way, in the presence of input target X, the target binding region of the constitutively inactive cgRNA is fully accessible and the effector handle is fully accessible, rendering the cgRNA active. The sequence of input target X is partially constrained by the sequence of target nucleic acid Y due to the common domain c*, and is further partially constrained by the sequence of the effector handle domain b*.

In some embodiments, the cgRNA is configured to conditionally perform a downstream function on the target nucleic acid (Y) by Mechanism 4 such that the input target (600) comprises from 3′ to 5′ a domain a* (615), and the domain b* (625), wherein the domain a (610) and the domain a* (615) are complementary to each other, and wherein the domain b (620) and the domain b* (625) are complementary to each other, and wherein the cgRNA is configured to be activated by the binding of the domain a (610) of the 5′ extension region (601) and domain a* (615) of the input target (600) to each other and domain b (620) of the 5′ extension region (601) and the domain b* (625) of the input target (600) to each other (FIG. 6).

In some embodiments, a cgRNA of Mechanism 4 (FIG. 6) can be employed. This can comprise a constitutively inactive cgRNA comprising a 5′ extension ab, a target binding region, an effector handle, and a terminator region that includes a terminator insert domain b*. In the absence of input target X, the 5′ extension domain b forms a hairpin with the terminator insert domain b*, sequestering the target binding region, thus rendering the cgRNA inactive. In the presence of input target X with sequence b*a* complementary to the 5′ extension domains ab, input target X hybridizes to the accessible toehold region a and domain b, exposing the previously sequestered target binding region. In this way, in the presence of input target X, the target binding region of the constitutively inactive cgRNA is fully accessible, rendering the cgRNA active. The sequence of input target X is fully independent of the sequence of the target nucleic acid Y. The loop formed in the inactive state may be partially structured, including formation of additional multiloops, interior loops, or bulge loops within the looped region. In some embodiments, the terminator insert region is at the 5′ end of the terminator region. In some embodiments, the terminator insert region is at the 3′ end of the terminator region. In some embodiments, the terminator insert region is contained somewhere between the 5′ and 3′ ends of the terminator region. In some embodiments, the canonical terminator region prior to addition of the terminator insert region, is the S. pyogenes terminator.

In some embodiments, the cgRNA is configured to conditionally perform a downstream function on the target nucleic acid (Y) by Mechanism 5 such that the input target (700) comprises from 3′ to 5′ a domain a* (715) and the domain b* (725), wherein the domain a (710) and the domain a* (715) are complementary to each other, and wherein the domain b (720) and the domain b* (725) are complementary to each other, and wherein the cgRNA is configured to be activated by the binding of the domain a (710) of the modified effector handle loop region (701) and domain a* (715) of the input target (700) to each other and domain b (720) of the modified effector handle loop region (701) and the domain b* (710) of the input target (710) to each other (FIG. 7).

In some embodiments, a cgRNA of Mechanism 5 (FIG. 7) can be employed. This can comprise a constitutively inactive cgRNA comprising a target binding region, an effector handle with a modified effector handle loop abc, and a terminator containing a terminator insert domain b*. In the absence of input target X, the modified effector handle loop domain b forms a hairpin with the terminator insert domain b*, disrupting the secondary structure of the effector handle, thus rendering the cgRNA inactive. In the presence of input target X with sequence b*a* complementary to the modified effector handle loop domains ab, input target X hybridizes to the accessible toehold region a and domain b, displacing the terminator insert domain and enabling formation of the effector handle, thus rendering the cgRNA active. The sequence of input target X is fully independent of the sequence of target nucleic acid Y. The loop formed in the inactive state may be partially structured, including formation of additional multiloops, interior loops, or bulge loops within the looped region. In some embodiments, the terminator insert region is at the 5′ end of the terminator region. In some embodiments, the terminator insert region is at the 3′ end of the terminator region. In some embodiments, the terminator insert region is contained somewhere between the 5′ and 3′ ends of the terminator region. In some embodiments, the canonical terminator region prior to addition of the terminator insert region, is the S. pyogenes terminator.

In some embodiments, the cgRNA is configured to conditionally perform a downstream function on the target nucleic acid (Y) by Mechanism 7 such that the input target (900) comprises from 3′ to 5′ a domain a* (915) and domain c (930), wherein the domain a (910) and the domain a* (915) are complementary to each other, and domain c (930) and the domain c* (935) are complementary to each other, and wherein the cgRNA is configured to be inactivated by the binding of the domain a* (915) of the input target (900) and domain a (910) of the 5′ extension region (901) to each other and the domain c (930) of the input target (900) and domain c* (935) of the 5′ extension region (901) to each other (FIG. 9).

In some embodiments, a cgRNA of Mechanism 7 can be employed. This can comprise a constitutively active cgRNA comprising a 5′ extension ac*bc, a target binding region b*d, a canonical effector handle, and an optional terminator region. In the absence of input target X, the 5′ extension ac*bc forms a hairpin and does not significantly interact with the target binding region or handle, preserving activity of the cgRNA and cgRNA/effector complex. In the presence of input target X with sequence ca* complementary to the ac* toehold and stem domains of the 5′ extension, input target X hybridizes to the toehold domain a and stem domain c* of the 5′ extension, exposing the previously sequestered loop domain b, which is then free to hybridize with domain b* of the target binding region. In this way, in the presence of input target X, the target binding region of the constitutively active cgRNA is sequestered, interfering with the capacity of the cgRNA/effector complex to bind target nucleic acid Y and rendering the cgRNA inactive. The target binding region may be fully sequestered in the presence of input target X for a zero-length domain d (i.e. where domain b* is the full target binding region). The sequence of input target X is fully independent of the sequence of target nucleic acid Y (i.e. unconstrained by the domains b*d).

In some embodiments, the cgRNA is configured to conditionally perform a downstream function on the target nucleic acid (Y) by Mechanism 8 such that the input target (1000) comprises from 3′ to 5′ a domain a* (1015) and domain b* (1025), wherein the domain a (1010) of the 5′ extension region (1001) and the domain a* (1015) of the input target (1000) are complementary to each other, and domain b (1020) of the modified effector handle loop region (1003C) and the domain b* (1025) of the input target (1000) are complementary to each other, and wherein the cgRNA is configured to be inactivated by the binding of the domain a (1010) of the 5′ extension region (1001) and the domain a* (1015) of the input target (1000) to each other and the domain b (1020) of the modified effector handle loop region (1003C) and the domain b* (1025) of the input target (1000) to each other (FIG. 10).

In some embodiments, a cgRNA of Mechanism 8 can be employed (FIG. 10). This can comprise a constitutively active cgRNA comprising a 5′ extension a, a target binding region, an effector handle containing a modified effector handle loop domain b, and an optional terminator region. In the absence of input target X, the 5′ extension a does not significantly interact with the target binding region or handle, allowing for activity of the cgRNA and cgRNA/effector complex. In the presence of input target X, with sequence b*a* complementary to the 5′ extension domain a and modified effector handle loop domain b, input target X hybridizes with the 5′ extension a and effector handle loop b, disrupting the secondary structure of the effector handle and sequestering the target binding region in a loop. In this way, in the presence of input target X, the target binding region of the constitutively active cgRNA is sequestered and effector handle structure is disrupted, interfering with the capacity of the cgRNA to be bound by effector and the capacity to bind target nucleic acid Y, thus rendering the cgRNA inactive. The sequence of input target X is fully independent of the sequence of target nucleic acid Y. The loop formed in the inactive state may be partially structured, including formation of additional multiloops, interior loops, or bulge loops within the looped region.

In some embodiments, the cgRNA is configured to conditionally perform a downstream function on the target nucleic acid (Y) by Mechanism 9 such that the input target (1100) comprises from 3′ to 5′ a domain a* (1115) and a domain b* (1125), wherein the domain a (1110) of the modified effector handle loop region (1103C) and the domain a* (1115) of the input target (1100) are complementary to each other, and domain b (1120) of the terminator insert region (1104A) and the domain b* (1125) of the input target (1100) are complementary to each other, and wherein the cgRNA is configured to be inactivated by the binding of the domain a (1110) of the modified effector handle loop region (1103C) and the domain a* (1115) of the input target (1100) to each other and the domain b (1120) of the terminator insert region (1104A) and the domain b* (1125) of the input target (1100) to each other (FIG. 11).

In some embodiments, a cgRNA of Mechanism 9 can be employed (FIG. 11). This can comprise a constitutively active cgRNA comprising a target binding region (1102), an effector handle (1103) containing a modified effector handle loop region (1103C) comprising a domain a (1110), and a terminator region (1104) containing a terminator insert domain (1104A) comprising a domain b (1120). In the absence of input target X (1100), the domain a of the modified effector handle loop and the domain b of the terminator insert region do not significantly interact with the target binding region or the effector handle, allowing for activity of the cgRNA and cgRNA/effector complex. In the presence of input target X (1100), with domain b* and domain a* that are complementary to the domain a of the modified effector handle loop and the domain b of the terminator insert region, respectively, input target X (1100) hybridizes with the modified effector handle loop and terminator insert region, disrupting the secondary structure of the effector handle. In this way, in the presence of input target X, the secondary structure of the cgRNA is disrupted, interfering with the capacity of the cgRNA to be bound by effector and the capacity to bind target nucleic acid Y, thus rendering the cgRNA inactive. The sequence of input target X is fully independent of the sequence of target nucleic acid Y. The loop formed in the inactive state may be partially structured, including formation of additional multiloops, interior loops, or bulge loops within the looped region.

In some embodiments, the cgRNA is configured to conditionally perform a downstream function on the target nucleic acid (Y) by Mechanism 10 such that the input target (1200) comprises from 3′ to 5′ a domain a* (1215), and wherein the domain a (1210) of the terminator insert region (1204A) and the domain a* (1215) of the input target (1200) are complementary to each other, and wherein the cgRNA is configured to be inactivated by the binding of the domain a (1210) of the terminator insert region (1204A) and the domain a* (1215) of the input target (1200) to each other (FIG. 12).

In some embodiments, a cgRNA of Mechanism 10 can be employed (FIG. 12). This can comprise a constitutively active cgRNA comprising a target binding region (1202), an effector handle (1203), and a terminator region (1204) that contains a terminator insert region (1204A). In the absence of input target X (1200), the domain a (1210) of the terminator insert region (1204A) does not significantly interact with the target binding region or effector handle, allowing for activity of the cgRNA and cgRNA/effector complex. In the presence of input target X (1200), with a domain a* (1215) that is complementary to the domain a (1210) of the terminator insert region (1204A), the input target X (1200) hybridizes with the terminator insert domain (1204A), disrupting the capacity of the cgRNA to mediate a downstream function on target nucleic acid Y, thus rendering the cgRNA inactive. The sequence of input target X (1200) is fully independent of the sequence of target nucleic acid Y. In some embodiments, the terminator insert region is at the 5′ end of the terminator region, immediately adjacent to the effector handle region. In some embodiments, the terminator insert region is at the 3′ end of the terminator region. In some embodiments, the terminator insert region is contained somewhere between the 5′ and 3′ ends of the terminator region. In some embodiments, the canonical terminator region prior to addition of the terminator insert region, is the S. pyogenes terminator.

In some embodiments, the cgRNA is configured to conditionally perform a downstream function on the target nucleic acid (Y) by Mechanism 11 such that the input target (1300) comprises from 3′ to 5′ a domain a* (1315), and wherein the domain a (1310) of the modified terminator loop region (1304C) and the domain a* (1315) of the input target (1300) are complementary to each other, and wherein the cgRNA is inactivated by the binding domain a (1310) of the modified terminator loop region (1304C) and the domain a* (1315) of the input target (1300) to each other (FIG. 13).

In some embodiments, a cgRNA of Mechanism 11 can be employed (FIG. 13). This can comprise a constitutively active cgRNA, comprising a target binding region (1302), an effector handle (1303), and a terminator region (1304) containing a modified terminator loop region (1304C) comprising domain a. In the absence of input target X (1300), the domain a (1310) of the modified terminator loop region (1304C) does not significantly interact with the target binding region (1302) or effector handle (1303), allowing for activity of the cgRNA and cgRNA/effector complex. In the presence of input target X (1300), with a domain a* (1315) that is complementary to the domain a (1310) of the modified terminator loop region (1304C), the input target X (1300) hybridizes with the modified terminator loop region (1304C), disrupting the canonical secondary structure of the cgRNA sufficiently to interfere with the capacity of the cgRNA to mediate a downstream function on target nucleic acid Y, thus rendering the cgRNA inactive. The sequence of input target X (1300) is fully independent of the sequence of the target nucleic acid Y. Input target X (1300) may be fully or partially complementary to the modified terminator loop, and may also include of full or partial sequence complementarity to other regions of the terminator in addition to the modified terminator loop.

In some embodiments, a conditional guide RNA (cgRNA) is provided. In some embodiments, the cgRNA comprises a target binding region and an effector handle. In some embodiments, the target binding region is configured to bind to a target nucleic acid (Y). In some embodiments, the cgRNA is configured to interact and form a complex with an RNA-guided effector. In some embodiments, the cgRNA is configured to interact and form a complex with an RNA-guided effector and configured to conditionally perform a downstream function on the target nucleic acid (Y) in an absence of an input target. In some embodiments, a cgRNA is configured to conditionally perform a downstream function on the target nucleic acid (Y) by Mechanism 6. In some embodiments, the cgRNA comprises from 5′ to 3′ the target binding region (802) comprising a domain a (810), the effector handle (803), and an optional terminator region (804), wherein the cgRNA is configured to be active, and wherein the cgRNA is inactivated by the binding of a domain a* (815) of an input target (800) and the domain a (810) of the target binding region (802) to each other.

In some embodiments, a cgRNA configured to conditionally perform a downstream function on the target nucleic acid (Y) by Mechanism 6 comprises the target binding region (802) comprising a domain a (810) 7-200 nt in length. In some embodiments the cgRNA comprises the target binding region (802) comprising a domain a (810) 30 nt in length or 12-100 nt in length. In some embodiments, domain a may be any length.

In some embodiments, a cgRNA of Mechanism 6 can be employed (FIG. 8). This can comprise a constitutively active cgRNA that comprises a canonical gRNA with target binding region (802) comprising a domain a (810), and an effector handle (803), and an optional terminator region (804). In the absence of input target X (800), the cgRNA is a canonical gRNA and is fully active. In the presence of input target X (800) with sequence complementary to the target binding region (802), input target X (800) hybridizes to the target binding region (802), sequestering the target binding region (800) and thereby interfering with the capacity of the cgRNA/effector complex to bind a target nucleic acid Y. The sequence of input target X (800) is fully determined by the sequence of the target nucleic acid Y (i.e. the sequence of the target binding region (802)). In some embodiments, the secondary structure of the complex of target binding region a and the target nucleic acid Y be partially structured, including formation of additional multiloops, interior loops, or bulge loops, and domain a (810) of the target binding region (802) may contain regions of non-complementarity with the target nucleic acid Y.

### Methods

In some embodiments, a method comprises providing one or more cgRNAs described herein, wherein the cgRNA changes its activity status depending on the presence or absence of an input target X. In some embodiments, the cgRNA is constitutively active in the absence of the input target X, and is conditionally inactivated by the presence of the input target X. In some embodiments, the cgRNA is constitutively inactive in the absence of the input target X, and is conditionally activated by the presence of the input target X. In some embodiments, a cgRNA with an active status interacts with an RNA-guided effector and performs a downstream function on a target nucleic acid Y.

In some embodiments, a method comprises providing one or more cgRNAs described herein, wherein the cgRNA interacts and forms a complex with an RNA-guided effector. In some embodiments, a method comprises providing one or more cgRNAs described herein, wherein the cgRNA interacts and forms a complex with an RNA-guided effector, and changing the activity status of the cgRNA depending upon a presence of an input target (X). In some embodiments, a method comprises providing one or more cgRNAs described herein, wherein the cgRNA interacts and forms a complex with an RNA-guided effector, and changing the activity status of the cgRNA depending upon an absence of an input target (X). In some embodiments, the method comprises binding of the complex to a specific target nucleic acid (Y).

In some embodiments, a method for conditionally performing a downstream function on a target nucleic acid is provided. In some embodiments, the method comprises providing a constitutively inactive conditional guide RNA (cgRNA). In some embodiments of the method, the cgRNA comprises an input target binding region, which binds to an input target, a target binding region, which binds to a target nucleic acid, and an effector handle region. In some embodiments of the method, in the presence of an RNA-guided effector, the cgRNA does not perform a downstream function on the target nucleic acid in the absence of the input target, but is conditionally activated to perform a downstream function on the target nucleic acid in the presence of the input target.

In some embodiments, a method comprises conditionally performing a downstream function by a cgRNA on a target nucleic acid based on Mechanism 1. In some embodiments, a method comprises providing an inactive cgRNA and conditionally performing a downstream function on a target nucleic acid by providing an input target, whereby a binding of the input target to the input target binding region of the cgRNA results in the cgRNA being conditionally activated to perform a downstream function on the target nucleic acid based on Mechanism 1.

In some embodiments, a method comprises conditionally performing a downstream function by a cgRNA on a target nucleic acid based on Mechanism 2. In some embodiments, a method comprises providing a constitutively inactive cgRNA and conditionally performing a downstream function on a target nucleic acid by providing an input target, whereby a binding of the input target to the input target binding region of the cgRNA results in the cgRNA being conditionally activated to perform a downstream function on the target nucleic acid based on Mechanism 2.

In some embodiments, a method comprises conditionally performing a downstream function by a cgRNA on a target nucleic acid based on Mechanism 3. In some embodiments, a method comprises providing a constitutively inactive cgRNA and conditionally performing a downstream function on a target nucleic acid by providing an input target, whereby a binding of the input target to the input target binding region of the cgRNA results in the cgRNA being conditionally activated to perform a downstream function on the target nucleic acid based on Mechanism 3.

In some embodiments, a method comprises conditionally performing a downstream function by a cgRNA on a target nucleic acid based on Mechanism 4. In some embodiments, a method comprises providing a constitutively inactive cgRNA and conditionally performing a downstream function on a target nucleic acid by providing an input target, whereby a binding of the input target to the input target binding region of the cgRNA results in the cgRNA being conditionally activated to perform a downstream function on the target nucleic acid based on Mechanism 4.

In some embodiments, a method comprises conditionally performing a downstream function by a cgRNA on a target nucleic acid based on Mechanism 5. In some embodiments, a method comprises providing a constitutively inactive cgRNA and conditionally performing a downstream function on a target nucleic acid by providing an input target, whereby a binding of the input target to the input target binding region of the cgRNA results in the cgRNA being conditionally activated to perform a downstream function on the target nucleic acid based on Mechanism 5.

In some embodiments, a method for conditionally performing a downstream function on a target nucleic acid is provided. In some embodiments, the method comprises providing a constitutively active conditional guide RNA (cgRNA). In some embodiments of the method, the cgRNA comprises an input target binding region, which binds to an input target, a target binding region, which binds to a target nucleic acid, and an effector handle region. In some embodiments of the method, the cgRNA interacts and forms a complex with an RNA-guided effector. In some embodiments of the method, the cgRNA interacts and forms a complex with an RNA-guided effector and performs a downstream function on the target nucleic acid in the absence of an input target. In some embodiments of the method, by a binding of the input target to the input target binding region of the cgRNA, the cgRNA is conditionally inactivated and ceases to perform a downstream function on the target nucleic acid.

In some embodiments, a method comprises conditionally performing a downstream function by a cgRNA on a target nucleic acid based on Mechanism 7. In some embodiments, a method comprises providing a constitutively active cgRNA and conditionally performing a downstream function on a target nucleic acid by providing an input target, whereby a binding of the input target to the cgRNA results in the cgRNA being conditionally inactivated based on Mechanism 7 and ceases to perform a downstream function on the target nucleic acid.

In some embodiments, a method comprises conditionally performing a downstream function by a cgRNA on a target nucleic acid based on Mechanism 8. In some embodiments, a method comprises providing a constitutively active cgRNA and conditionally performing a downstream function on a target nucleic acid by providing an input target, whereby a binding of the input target to the cgRNA results in the cgRNA being conditionally inactivated based on Mechanism 8 and ceases to perform a downstream function on the target nucleic acid.

In some embodiments, a method comprises conditionally performing a downstream function by a cgRNA on a target nucleic acid based on Mechanism 9. In some embodiments, a method comprises providing a constitutively active cgRNA and conditionally performing a downstream function on a target nucleic acid by providing an input target, whereby a binding of the input target to the cgRNA results in the cgRNA being conditionally inactivated based on Mechanism 9 and ceases to perform a downstream function on the target nucleic acid.

In some embodiments, a method comprises conditionally performing a downstream function by a cgRNA on a target nucleic acid based on Mechanism 10. In some embodiments, a method comprises providing a constitutively active cgRNA and conditionally performing a downstream function on a target nucleic acid by providing an input target, whereby a binding of the input target to the cgRNA results in the cgRNA being conditionally inactivated based on Mechanism 10 and ceases to perform a downstream function on the target nucleic acid.

In some embodiments, a method comprises conditionally performing a downstream function by a cgRNA on a target nucleic acid based on Mechanism 11. In some embodiments, a method comprises providing a constitutively active cgRNA and conditionally performing a downstream function on a target nucleic acid by providing an input target, whereby a binding of the input target to the cgRNA results in the cgRNA being conditionally inactivated based on Mechanism 11 and ceases to perform a downstream function on the target nucleic acid.

### Additional Embodiments

Any one or more of the embodiments below can be combined or substituted into any one of the methods and/or mechanisms discussed above.

In some embodiments of the cgRNAs described herein, the effector handle region is configured to interact and form a complex with an effector protein selected from the group consisting of Cas9, dCas9, C2C2, Cas13d, any protein fusions or derivatives thereof, any RNA-guided effector (e.g., CRISPR) protein or protein complex, any protein from a similar pathway, and any protein the mediates a downstream function on a target nucleic acid in complex with a cgRNA with an active status.

In some embodiments of the cgRNAs described herein, the cgRNA comprises one or more chemical modifications that alter one or more of degradation properties, affinity, biological activity, and delivery properties of the cgRNA. In some embodiments of the cgRNAs described herein, the one or more chemical modifications is selected from the group consisting of arabino nucleic acids (ANA), locked nucleic acids (LNA), peptide nucleic acids (PNA), phosphoroamidate DNA analogues, phosphorodiamidate morpholino oligomers (PMO), cyclohexene nucleic acids (CeNA), tricycloDNA (tcDNA), bridged nucleic acids (BNA), phosphorothioate modification, 2′-fluoro (2′-F) modification, 2′-fluoroarabino (2′-FANA) modification, 2′O-Methyl (2′O-Me) modification, and 2′O-(2-methoxyethyl) (2′O-MOE) modification.

In some embodiments of the cgRNAs described herein, a sequence of the cgRNA may be a subsequence of a longer RNA, DNA, or another polymer capable of base-pairing.

In some embodiments, complementary domains (e.g., complementary domains within a cgRNA, or complementary domains between a cgRNA and an input target, or complementary domains between a cgRNA and a target nucleic acid) may contain one or more mismatches. In some embodiments, the number of mismatches can range from 1 to about 20. In some embodiments, the number of mismatches can range from 5 to about 20. In some embodiments, the number of mismatches can be 1, 2, 3, 4, 5, 6, 7, 8, 9, 10, 11, 12, 13, 14, 15, 16, 17, 18, 19, or 20. In some embodiments, complementary domains incorporating one or more mismatches will form secondary structures that may include, without limitation, one or more of loops, multiloops or bulges due to base-pairing interactions within or between any of the cgRNA domains, between cgRNA domains and input target domains, or between cgRNA domains and target nucleic acid domains.

In some embodiments of the cgRNAs described herein, the cgRNA may be expressed in the cells, living organisms or artificial settings in which it interacts with effector, input, and/or target, or may be synthesized exogenously and introduced.

In some embodiments of the cgRNAs described herein, the cgRNA may conditionally perform a downstream function on a target nucleic acid in one or more of living organisms, ecosystems, tissue extracts, cell lysates, or artificial systems of reconstituted biological components.

In some embodiments of the cgRNAs described herein, a sequence of input target may be fully constrained, partially constrained, or fully unconstrained by the sequence of target nucleic acid.

In some embodiments of the cgRNAs described herein, a sequence of an input target may be a subsequence of a longer RNA, DNA, or another polymer capable of base-pairing.

In some embodiments of the cgRNAs described herein, the target nucleic acid may be RNA, DNA, or another polymer capable of base-pairing, coding or non-coding, endogenous or exogenous.

In some embodiments of the cgRNAs described herein, the RNA-guided effector is selected from the group consisting of Cas9, dCas9, C2C2, protein fusions or derivatives thereof, RNA-guided effector protein (e.g., RNA-guided CRISPR effector) or protein complex, any protein from a similar pathway, and any protein the mediates a downstream function on a target nucleic acid in complex with a cgRNA with an active status.

In some embodiments of the methods described herein, the downstream function is selected from the group consisting of activating an expression of the target nucleic acid, silencing an expression of the target nucleic acid, editing the target nucleic acid, and binding the target nucleic acid.

In some embodiments of the methods described herein, changing the activity status of the cgRNA results in a conditional increase or a conditional decrease in the downstream function relative to a basal level of a cgRNA-mediated activity on the target nucleic acid. In some embodiments, the conditional increase ranges from about 2 fold to about 200 fold. In some embodiments, the conditional increase ranges from about 5 fold to about 500 fold. In some embodiments, the conditional increase is about 1.5, 2, 5, 10, 50, 100, 200, 300, 400, 500, 600 700, 800, 900, or 1000 fold, or a value within a range defined by any two of the aforementioned values. In some embodiments, the conditional decrease ranges from about 2 fold to about 200 fold. In some embodiments, the conditional decrease ranges from about 5 fold to about 500 fold. In some embodiments, the conditional decrease is about 1.5, 2, 5, 10, 50, 100, 200, 300, 400, 500, 600 700, 800, 900, or 1000 fold, or a value within a range defined by any two of the aforementioned values.

In some embodiments, the effector handle region is configured to interact and form a complex with an effector protein selected from the group consisting of Cas9, dCas9, C2C2, Cas13d, any protein fusions or derivatives thereof, any RNA-guided effector (e.g., RNA-guided CRISPR effector) protein or protein complex, any protein from a similar pathway, and any protein the mediates a downstream function on a target nucleic acid in complex with a cgRNA with an active status.

In some embodiments, the cgRNA comprises one or more chemical modifications that alter one or more of degradation properties, affinity, biological activity, and delivery properties of the cgRNA. In some embodiments, the one or more chemical modifications is selected from the group consisting of arabino nucleic acids (ANA), locked nucleic acids (LNA), peptide nucleic acids (PNA), phosphoroamidate DNA analogues, phosphorodiamidate morpholino oligomers (PMO), cyclohexene nucleic acids (CeNA), tricycloDNA (tcDNA), bridged nucleic acids (BNA), phosphorothioate modification, 2′-fluoro (2′-F) modification, 2′-fluoroarabino (2′-FANA) modification, 2′O-Methyl (2′O-Me) modification, and 2′O-(2-methoxyethyl) (2′O-MOE) modification.

In some embodiments, a sequence of the cgRNA may be a subsequence of a longer RNA, DNA, or another polymer capable of base-pairing.

In some embodiments, complementary domains (e.g., complementary domains within a cgRNA, or complementary domains between a cgRNA and an input target, or complementary domains between a cgRNA and a target nucleic acid) may contain one or more mismatches. In some embodiments, the number of mismatches can be 1, 2, 3, 4, 5, 6, 7, 8, 9, 10, or 15, or 20. In some embodiments, complementary domains incorporating one or more mismatches will form secondary structures that may include, without limitation, one or more of loops, multiloops or bulges due to base-pairing interactions within or between any of the cgRNA domains, between cgRNA domains and input target domains, or between cgRNA domains and target nucleic acid domains.

In some embodiments, the cgRNA may be expressed in the cells, living organisms or artificial settings in which it interacts with effector, input, and/or target, or may be synthesized exogenously and introduced.

In some embodiments, the cgRNA may conditionally perform a downstream function on a target nucleic acid in one or more of living organisms, ecosystems, tissue extracts, cell lysates, or artificial systems of reconstituted biological components.

In some embodiments, a sequence of input target may be fully constrained, partially constrained, or fully unconstrained by the sequence of target nucleic acid.

In some embodiments, a sequence of input target may be a subsequence of a longer RNA, DNA, or another polymer capable of base-pairing.

In some embodiments, the target nucleic acid may be RNA, DNA, or another polymer capable of base-pairing, coding or non-coding, endogenous or exogenous.

In some embodiments, the RNA-guided effector is selected from the group consisting of Cas9, dCas9, C2C2, protein fusions or derivatives thereof, RNA-guided effector (e.g., RNA-guided CRISPR effector) protein or protein complex, any protein from a similar pathway, and any protein the mediates a downstream function on a target nucleic acid in complex with a cgRNA with an active status.

In some embodiments, a sequence of the target nucleic acid Y can be chosen to determine the target nucleic acid, and the sequence of input target X (which may be fully constrained, partially constrained, or unconstrained by sequence Y) can be chosen to control the scope of modulation (space, time, cell, tissue, organ, organism, ecosystem, etc). In some embodiments, the inactive state of the cgRNA is achieved with secondary or tertiary structural elements that interfere with its capacity to mediate interaction between the effector protein and the target nucleic acid Y, for example by a) inhibiting the formation of the cgRNA/effector complex, b) inhibiting the association of target nucleic acid Y and the cgRNA/effector complex, and/or c) inhibiting the activity of the effector in the cgRNA/effector/target complex. In some embodiments, in the active state, the cgRNA presents an accessible target-binding region, and any structural modifications to the cgRNA must preserve the structural and sequence requirements for formation and activity of the cgRNA/effector complex.

FIG. 3-FIG. 13 contain cgRNA Mechanisms 1-11. In Mechanisms 1-5, a constitutively inactive cgRNA is conditionally activated by the presence of an input target. In Mechanisms 6-11, a constitutively active cgRNA is conditionally inactivated by the presence of an input target.

In some embodiments, the secondary structure of a cgRNA (e.g., Mechanisms 1-11), or of a cgRNA/input target complex may contain interior loops, multiloops, or bulge loops due to mismatches between any of the complementary domains of the cgRNA or input target.

In some embodiments, the effector handle region is configured to interact and form a complex with an effector protein selected from the group consisting of Cas9, dCas9, C2C2, Cas13d, any protein fusions or derivatives thereof, any RNA-guided effector (e.g., RNA-guided CRISPR effector) protein or protein complex, any protein from a similar pathway, and any protein the mediates a downstream function on a target nucleic acid in complex with a cgRNA with an active status.

In some embodiments, the Mechanisms 1-11 may be implemented for the conditional interaction between target nucleic acid and Cas9, dCas9, any protein fusions or derivatives thereof. The handle region of the cgRNA may also be modified for the conditional interaction between target nucleic acid and C2C2, Cas13d, any RNA-guided effector (e.g., RNA-guided CRISPR effector) protein or protein complex, any protein from a similar pathway, or any protein the mediates a downstream function on a target nucleic acid in complex with a cgRNA with an active status.

In some embodiments, the cgRNA may be a single nucleic acid strand or a complex of nucleic acid strands.

In some embodiments, the sequence of the cgRNA may be a subsequence of a longer RNA, DNA, or another polymer capable of base-pairing.

In some embodiments, the cgRNA may be constitutively active, i.e. capable of mediating an interaction between an RNA-guided effector and a target nucleic acid Y in the absence of input target X with reduced ability to mediate interaction between the RNA-guided effector and the target nucleic acid Y in the presence of input target X, or constitutively inactive, i.e. capable of mediating interaction between an RNA-guided effector and the target nucleic acid Y in the presence of input target X with reduced ability to mediate interaction between the RNA-guided effector and the target nucleic acid Y in the absence of input target X.

In some embodiments, the input target X may be RNA, DNA, or another polymer capable of base-pairing, coding or non-coding, endogenous or exogenous.

In some embodiments, the target nucleic acid Y may be RNA, DNA, or another polymer capable of base-pairing, coding or non-coding, endogenous or exogenous.

In some embodiments, the sequence of input target X may be fully constrained, partially constrained, or fully unconstrained by the sequence of target nucleic acid Y.

In some embodiments, the sequence of input target X may be a subsequence of a longer RNA, DNA, or another polymer capable of base-pairing.

In some embodiments, the secondary structure of input target X may contain loops, multiloops or bulges.

In some embodiments, the cgRNA may conditionally mediate interaction between RNA-guided effector and target nucleic acid Y in cultured cells, living organisms, ecosystems, tissue extracts, cell lysates, or artificial systems of reconstituted biological components.

In some embodiments, the inactive state of the cgRNA may be achieved by inhibiting the formation of the cgRNA/effector complex, by inhibiting the association of target nucleic acid Y and the cgRNA/effector complex, and/or by inhibiting the activity of the RNA-guided effector in the cgRNA/effector/target complex, or by other means.

In some embodiments, the inhibition of cgRNA/effector, cgRNA/target or cgRNA/effector/target interaction may be achieved by intra- or inter-molecular hybridization, and/or modifications to cgRNA structure or sequence.

The cgRNA may be expressed in the cells, living organisms or artificial settings in which it interacts with effector, input, and/or target, or may be synthesized exogenously and introduced.

In some embodiments, the cgRNA may be chemically modified so as to alter degradation properties, affinity, biological activity, and/or delivery properties (e.g., variants including arabino nucleic acids (ANA), locked nucleic acids (LNA), peptide nucleic acids (PNA), phosphoroamidate DNA analogues, phosphorodiamidate morpholino oligomers (PMO), cyclohexene nucleic acids (CeNA), tricycloDNA (tcDNA), bridged nucleic acids (BNA), phosphorothioate modification, 2′-fluoro (2′-F) modification, 2′-fluoroarabino (2′-FANA) modification, 2′O-Methyl (2′O-Me) modification, or 2′O-(2-methoxyethyl) (2′O-MOE) modification).

In some embodiments, the cgRNA may incorporate elements to facilitate monitoring of localization within a sample via chemical modification (e.g., modification with fluorophores, chromophores, fluorescent quenchers, radiolabeled nucleotides) or by incorporation of nucleotides specific for the binding of fluorescent or other reporter proteins, or by incorporation of an aptamer-based fluorescent biosensor.

In some embodiments, intracellular delivery of the cgRNA may be promoted by backbone modification, such as phosphorothioate modification, and/or by incorporation of ligands to enable uptake by the cell, such as cell-penetrating peptides or small-molecule targeting ligands.

### EXAMPLES

The examples provided herein are not intended to be limiting.

### Example 1

This Example shows in vitro cleavage of specific target nucleic acids by specific constitutively active cgRNAs conditionally inactivated by specific input targets (Mechanism 8, Constitutively Active Splinted Switch A).

FIG. 14A demonstrates Mechanism 8 (Constitutively Active Splinted Switch A) performing conditional dsDNA cleavage in vitro using wildtype recombinant Cas9. Lanes left to right: 1) dsDNA target nucleic acid Y with recombinant Cas9 only, 2) dsDNA target nucleic acid Y with recombinant Cas9 and a canonical gRNA positive control, 3) dsDNA target nucleic acid Y with recombinant Cas9 and a constitutively active cgRNA (15 nt 5′ extension, 15 nt modified effector handle loop), 4) dsDNA target nucleic acid Y with recombinant Cas9 and a constitutively active cgRNA (15 nt 5′ extension, 15 nt modified effector handle loop) and 30 nt RNA input target X (2:1 ratio of input target to cgRNA). In the absence of input target X, the constitutively active cgRNA mediates cleavage of dsDNA target nucleic acid Y by Cas9 with activity comparable to the canonical positive control gRNA. In the presence of input target X, no cleavage of dsDNA target Y is observed.

FIG. 14B demonstrates the orthogonal operation of Mechanism 8 with a library of 4 constitutively active orthogonal cgRNAs (A, B, C, D) with 15 nt 5′ extension, 15 nt modified effector handle loop. Input target A conditionally inactivates cgRNA A, but not cgRNAs B, C, D. Input target B conditionally inactivates cgRNA B, but not cgRNAs A, C, D. Input target C conditionally inactivates cgRNA C, but not cgRNAs A, B, D. Input target D conditionally inactivates cgRNA D, but not cgRNAs A, B, C. All lanes contain dsDNA target nucleic acid Y, recombinant Cas9, cgRNA, and 30 nt RNA input target (10:1 ratio of input target to cgRNA; input target A, B, C or D as indicated). In the absence of cognate input target (viz. cgRNA A+input target B, C, or D; cgRNA B+input target A, C, or D; cgRNA C+input target A, B, or D; and cgRNA D+input target A, B, or C), the constitutively active cgRNA mediates cleavage of dsDNA target nucleic acid Y. Each of the 4 cgRNAs are rendered inactive in the presence of the corresponding cognate input target (viz. cgRNA A+input target A, cgRNA B+input target B, cgRNA C+input target C, cgRNA D+input target D), with no observed cleavage activity in the presence of cognate input target.

### Example 2

This example describes silencing of expression of specific target nucleic acids by specific constitutively active cgRNAs, conditionally inactivated by specific input targets in E. coli expressing RNA-guided effector dCas9 (Mechanism 9, Constitutively Active Splinted Switch B).

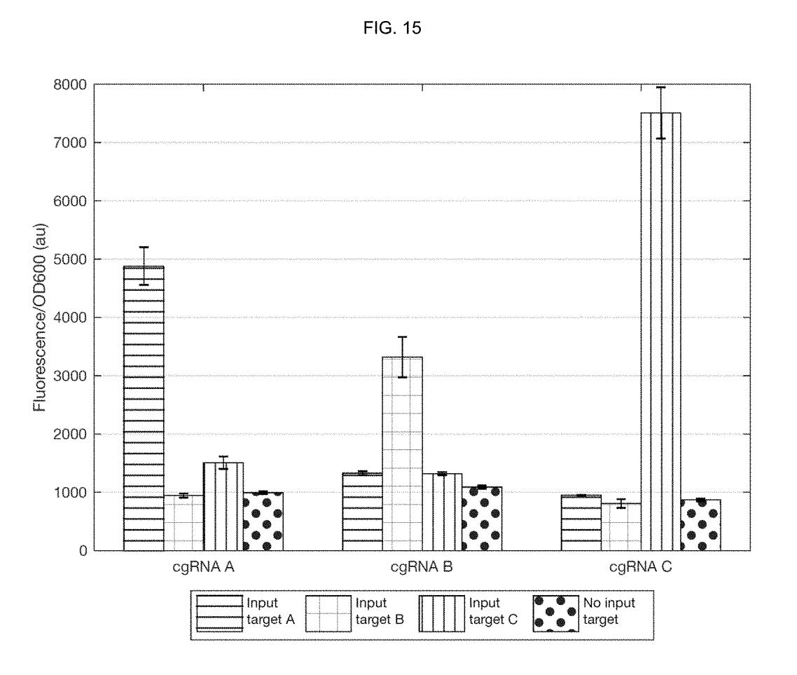

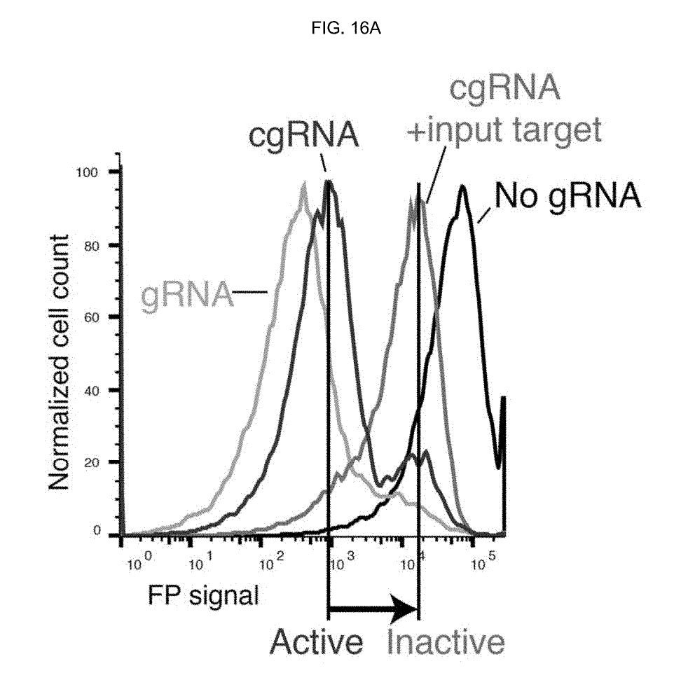

FIG. 15 demonstrates orthogonal operation of Mechanism 9 (Constitutively Active Splinted Switch B) in E. coli expressing effector dCas9 for a library of 3 constitutively active orthogonal cgRNAs (A, B, C) with 35 nt modified effector handle loop and a 35 nt terminator insert domain. Input target A conditionally inactivates cgRNA A, but not cgRNAs B, C. Input target B conditionally inactivates cgRNA B, but not cgRNAs A, C. Input target C conditionally inactivates cgRNA C, but not cgRNAs A, B. Each bar corresponds to A600 normalized fluorescence of an E. coli. strain expressing: one of three cgRNA sequences (cgRNA A, left cluster; cgRNA B, center cluster; cgRNA C, right cluster), one of three input target sequences (input target A, input target B, input target C) or no input target, RNA-guided effector dCas9, and a genomically incorporated fluorescent protein. Data are measured by microplate fluorescence ˜16h post induction of dCas9. In the absence of cognate input target (viz. cgRNA A+input target B, C, or no input target; cgRNA B+input target A, C, or no input target; cgRNA C+input target A, B, or no input target), the constitutively active cgRNA mediates silencing of expression of DNA target nucleic acid Y by dCas9. Normalized fluorescence is significantly higher in strains in which cgRNA and cognate input target are co-expressed (viz. cgRNA A+input target A, cgRNA B+input target B, cgRNA C+input target C), with non-cognate cgRNA/input target combinations resulting in normalized fluorescence comparable to the no input target condition. Error bars are estimate of standard deviation between 3 replicates.

### Example 3

This example describes silencing of expression of specific target nucleic acids by specific constitutively active cgRNAs, conditionally inactivated by specific input targets in E. coli expressing RNA-guided effector dCas9 (Constitutively Active Terminator Switch: Mechanism 10).

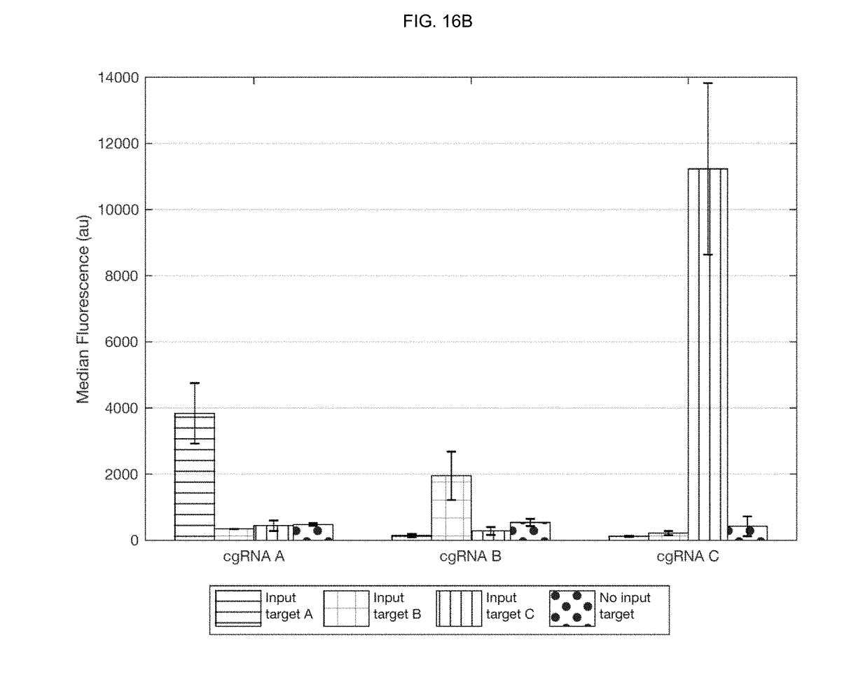

FIG. 16A demonstrates Mechanism 10 (Constitutively active Terminator Switch) performing conditional silencing of a target nucleic acid Y in E. coli expressing RNA-guided effector dCas9. The target nucleic acid Y is a genomic DNA for a fluorescent protein. Conditional silencing of expression of the target nucleic acid Y is assayed using flow cytometry to monitor expression of the fluorescent protein reporter. Expression of a short RNA input target X causes greater than one-order-of-magnitude increase in fluorescence (corresponding to the cgRNA switching from active to inactive). gRNA and cgRNA contain identical target binding regions corresponding to a constitutively expressed fluorescent protein DNA (target nucleic acid Y). In the absence of input target X, the constitutively active cgRNA with 44 nt modified terminator loop domain mediates transcriptional repression of DNA target Y by dCas9 with corresponding fluorescent signal comparable to the canonical positive control gRNA. A greater than 1 order of magnitude increase in fluorescence is observed for the experimental strain expressing input target X (input target+cgRNA).

FIG. 16B demonstrates orthogonal operation of Mechanism 10 with a library of 3 constitutively active orthogonal cgRNAs (A, B, C) with 44 nt modified terminator insert domain. Input target A conditionally inactivates cgRNA A, but not cgRNAs B, C. Input target B conditionally inactivates cgRNA B, but not cgRNAs A, C. Input target C conditionally inactivates cgRNA C, but not cgRNAs A, B. Each bar corresponds to median normalized fluorescence of an E. coli. strain expressing: one of three cgRNA sequences (cgRNA A, left cluster; cgRNA B, center cluster; cgRNA C, right cluster), one of three input target sequences (Input target A, Input target B, Input target C) or no input target, RNA-guided effector dCas9, and a genomically incorporated fluorescent protein. Data are measured by flow cytometry ˜14h post induction of dCas9. Normalized fluorescence is significantly higher in strains in which cgRNA and cognate input target are co-expressed (viz. cgRNA A+Input target A, cgRNA B+Input target B, cgRNA C+Input target C), with non-cognate cgRNA/input target combinations resulting in normalized fluorescence comparable to the no input target condition. Error bars are estimate of standard deviation between 2 replicates.

### Example 4

This example describes silencing of expression of specific target nucleic acids by specific constitutively inactive cgRNAs, conditionally activated by specific input targets in E. coli expression RNA-guided effector dCas9 (Constitutively Inactive Toehold Switch: Mechanism 1).

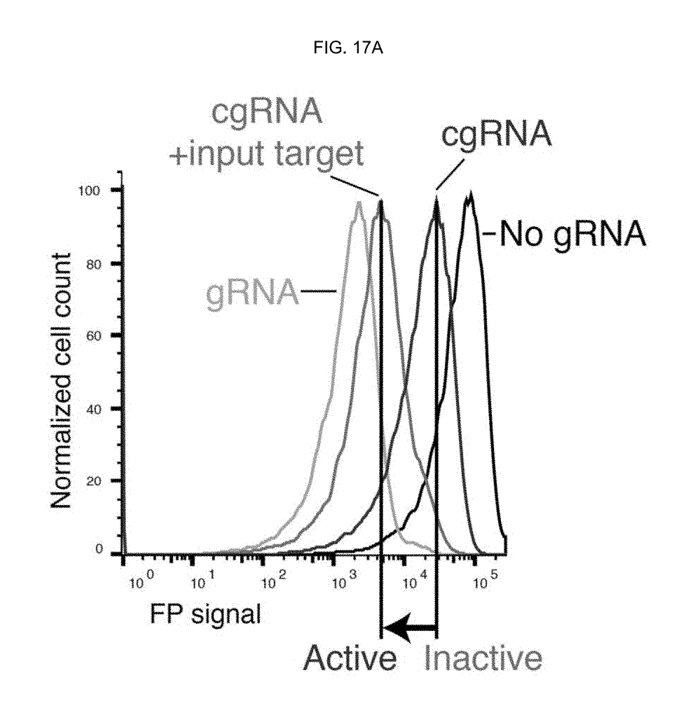

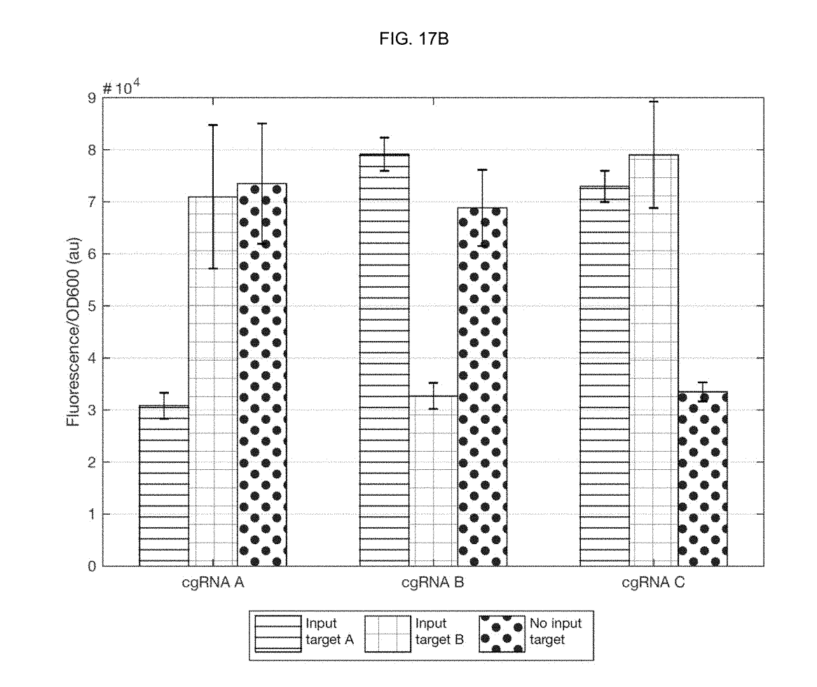

FIG. 17A demonstrates Mechanism 1 (Constitutively inactive Toehold Switch) performing conditional gene silencing in E. coli expressing RNA-guided effector dCas9. The target nucleic acid Y is a genomic DNA for a fluorescent protein. Conditional silencing of expression of the target nucleic acid Y is assayed using flow cytometry to monitor expression of the fluorescent protein reporter. Expression of a short RNA input target X causes an approximately one-order-of-magnitude decrease in fluorescence (corresponding to the cgRNA switching from inactive to active). gRNA and cgRNA contain identical target binding regions corresponding to a constitutively expressed fluorescent protein DNA (target nucleic acid Y). In a strain expressing the constitutively inactive cgRNA (20 nt target binding region sequestered in 20 nt stem, 8 nt hairpin loop, 15 nt toehold domain) but not input target X, target nucleic acid Y is expressed. In the presence of input target X, the input target activates the constitutively inactive cgRNA, which thereby mediates silencing of expression of DNA target Y by dCas9 (input target+cgRNA), corresponding to approximately an order of magnitude decrease in fluorescence.

FIG. 17B demonstrates orthogonal operation of Mechanism 1 with a library of 3 constitutively inactive cgRNAs (A, B, C). Input target A conditionally activates cgRNA A, but not cgRNAs B, C. Input target B conditionally activates cgRNA B, but not cgRNAs A, C. Input target C conditionally activates cgRNA C, but not cgRNAs A, B. Each bar corresponds to A600 normalized fluorescence of an E. coli. strain expressing: one of three cgRNA sequences (cgRNA A, left cluster; cgRNA B, center cluster; cgRNA C, right cluster), one of three input target sequences (Input target A, Input target B, Input target C), RNA-guided effector dCas9, and a genomically incorporated fluorescent protein. Data are measured by microplate fluorescence ˜8h post induction of dCas9. Normalized fluorescence is significantly lower in strains in which cgRNA and cognate input target are co-expressed (viz. cgRNA A+Input target A, cgRNA B+Input target B, cgRNA C+Input target C) as compared to non-cognate cgRNA/input target combinations. Error bars are estimate of standard deviation between 3 replicates.

### REFERENCES

All references are incorporated by reference in their entireties.
Abudayyeh, Omar O., Jonathan S. Gootenberg, Silvana Konermann, Julia Joung, Ian M. Slaymaker, David B. T. Cox, Sergey Shmakov, Kira S. Makarova, Ekaterina Semenova, Leonid Minakhin, Konstantin Severinov, Aviv Regev, Eric S. Lander, Eugene V. Koonin, and Feng Zhang. 2016. ‘C2c2 is a single-component programmable RNA-guided RNA-targeting CRISPR effector’, Science.
Barrangou, Rodolphe, Christophe Fremaux, Hdlene Deveau, Melissa Richards, Patrick Boyaval, Sylvain Moineau, Dennis A. Romero, and Philippe Horvath. 2007. ‘CRISPR Provides Acquired Resistance Against Viruses in Prokaryotes’, Science, 315: 1709-12.
Chen, Baohui, Luke A Gilbert, Beth A Cimini, Joerg Schnitzbauer, Wei Zhang, Gene-Wei Li, Jason Park, Elizabeth H Blackburn, Jonathan S Weissman, Lei S Qi, and Bo Huang. 2013. ‘Dynamic Imaging of Genomic Loci in Living Human Cells by an Optimized CRISPR/Cas System’, Cell, 155: 1479-91.
Cong, Le, F. Ann Ran, David Cox, Shuailiang Lin, Robert Barretto, Naomi Habib, Patrick D. Hsu, Xuebing Wu, Wenyan Jiang, Luciano A. Marraffini, and Feng Zhang. 2013. ‘Multiplex Genome Engineering Using CRISPR/Cas Systems’, Science, 339: 819-23.
DiCarlo, James E, Alejandro Chavez, Sven L Dietz, Kevin M Esvelt, and George M Church. 2015. ‘RNA-guided gene drives can efficiently bias inheritance in wild yeast’, bioRxiv.
Gilbert, Luke A, Matthew H Larson, Leonardo Morsut, Zairan Liu, Gloria A Brar, Sandra E Torres, Noam Stern-Ginossar, Onn Brandman, Evan H Whitehead, Jennifer A Doudna, Wendell A Lim, Jonathan S Weissman, and Lei S Qi. 2013. ‘CRISPR-Mediated Modular RNA-Guided Regulation of Transcription in Eukaryotes’, Cell, 154: 442-51.
Horvath, Philippe, and Rodolphe Barrangou. 2010. ‘CRISPR/Cas, the Immune System of Bacteria and Archaea’, Science, 327: 167-70.
Jinek, Martin, Krzysztof Chylinski, Ines Fonfara, Michael Hauer, Jennifer A. Doudna, and Emmanuelle Charpentier. 2012. ‘A Programmable Dual-RNA-Guided DNA Endonuclease in Adaptive Bacterial Immunity’, Science, 337: 816-21.
Mali, Prashant, Luhan Yang, Kevin M. Esvelt, John Aach, Marc Guell, James E. DiCarlo, Julie E. Norville, and George M. Church. 2013. ‘RNA-Guided Human Genome Engineering via Cas9’, Science, 339: 823-26.
Qi, Lei S, Matthew H Larson, Luke A Gilbert, Jennifer A Doudna, Jonathan S Weissman, Adam P Arkin, and Wendell A Lim. 2013. ‘Repurposing CRISPR as an RNA-Guided Platform for Sequence-Specific Control of Gene Expression’, Cell, 152: 1173-83.
Sander, Jeffry D., and J. Keith Joung. 2014. ‘CRISPR-Cas systems for editing, regulating and targeting genomes’, Nat Biotech, 32: 347-55.

1. (canceled)

2. (canceled)

3. A cgRNA comprising:
an input target binding region, configured to bind to an input target;
a target binding region, configured to bind to a target nucleic acid; and
an effector handle region,
wherein the cgRNA is configured to conditionally perform a downstream function on the target nucleic acid in a presence of the input target and an RNA-guided effector.

4. A cgRNA comprising:
an input target binding region, configured to bind to an input target;
a target binding region, configured to bind to a target nucleic acid; and
an effector handle region,
wherein, the cgRNA is configured to interact and form a complex with an RNA-guided effector, and
wherein the complex is configured to conditionally perform a downstream function on the target nucleic acid in an absence of the input target.

5. The cgRNA of claim 3, comprising from 5′ to 3′ the 5′ extension region comprising a domain a, a domain b, and a domain c, the target binding region comprising a domain b*, and a domain d, the effector handle region, wherein the domain b of the 5′ extension region and the domain b* of the target binding region are complementary to each other, and wherein the cgRNA is configured to be inactive by the binding of the domain b of the 5′ extension region and the domain b* of the target binding region to each other.

6. (canceled)

7. (canceled)

8. (canceled)

9. (canceled)

10. (canceled)

11. (canceled)

12. The cgRNA of claim 4, comprising from 5′ to 3′ the target binding region, a first partial sequence of the effector handle, a modified effector handle loop region comprising a domain a, a second partial sequence of the effector handle, a terminator insert region comprising a domain b, and a terminator region, wherein the cgRNA is configured to be active.

13. The cgRNA of claim 4, comprising from 5′ to 3′ the target binding region, the effector handle, a terminator insert region comprising a domain a, and a terminator region, wherein the cgRNA is configured to be active.

14. (canceled)

15. The cgRNA of claim 5, wherein the input target comprises from 3′ to 5′ a domain a* and the domain b*, and wherein the domain a and the domain a* are complementary to each other, and the domain b and the domain b* are complementary to each other, and wherein the cgRNA is configured to be activated by the binding of the domain a of the 5′ extension region and domain a* of the input target to each other and domain b of the 5′ extension region and the domain b* of the input target to each other.

16. (canceled)

17. (canceled)

18. (canceled)

19. (canceled)

20. (canceled)

21. (canceled)

22. The cgRNA of claim 12, wherein the input target comprises from 3′ to 5′ a domain a* and a domain b*, wherein the domain a of the modified effector handle loop region and the domain a* of the input target are complementary to each other, and domain b of the terminator insert region and the domain b* of the input target are complementary to each other, and wherein the cgRNA is configured to be inactivated by the binding of the domain a of the modified effector handle loop region and the domain a* of the input target to each other and the domain b of the terminator insert region and the domain b* of the input target to each other.

23. The cgRNA of claim 13, wherein the input target comprises from 3′ to 5′ a domain a*, and wherein the domain a of the terminator insert region and the domain a* of the input target are complementary to each other, and wherein the cgRNA is configured to be inactivated by the binding of the domain a of the terminator insert region and the domain a* of the input target to each other.

24. (canceled)

25. A method comprising providing a conditional guide RNA (cgRNA), wherein the cgRNA changes its activity status depending upon a presence or an absence of an input target.

26. The method of claim 25, further comprising forming a complex with an RNA-guided effector and binding a specific target nucleic acid.

27. The method of claim 26 for conditionally performing a downstream function on a target nucleic acid, the method comprising:
providing an inactive conditional guide RNA (cgRNA) comprising:
an input target binding region, configured to bind to an input target;
a target binding region, configured to bind to the target nucleic acid; and
an effector handle region,
conditionally performing a downstream function on the target nucleic acid by providing an input target and an RNA-guided effector, wherein by a binding of the input target to the cgRNA, the cgRNA is activated to perform a downstream function on the target nucleic acid.

28. The method of claim 27, comprising:
providing an inactive conditional guide RNA (cgRNA) according to claim 5,
conditionally performing a downstream function on the target nucleic acid by providing an input target according to claim 15 and an RNA-guided effector, wherein by a binding of the input target to the cgRNA, the cgRNA is activated to perform a downstream function on the target nucleic acid.

29. (canceled)

30. (canceled)

31. (canceled)

32. (canceled)

33. The method of claim 26, for conditionally performing a downstream function on a target nucleic acid, the method comprising:
providing an active conditional guide RNA (cgRNA) comprising:
an input target binding region, configured to bind to an input target;
a target binding region, configured to bind to the target nucleic acid; and
an effector handle region,
wherein, the cgRNA is configured to interact and form a complex with an RNA-guided effector; and
conditionally performing a downstream function on the target nucleic acid by providing an input target, wherein by a binding of the input target to the cgRNA, the cgRNA ceases to perform a downstream function on the target nucleic acid.

34. (canceled)

35. (canceled)

36. The method of claim 33, comprising:
providing a conditional guide RNA (cgRNA) comprising from 5′ to 3′ the target binding region, a first partial sequence of the effector handle, a modified effector handle loop region comprising a domain a, a second partial sequence of the effector handle, a terminator insert region comprising a domain b, and a terminator region, wherein the cgRNA is configured to be active; and
conditionally performing a downstream function on the target nucleic acid by providing an input target comprising from 3′ to 5′ a domain a* and a domain b*, wherein the domain a of the modified effector handle loop region and the domain a* of the input target are complementary to each other, and domain b of the terminator insert region and the domain b* of the input target are complementary to each other, and wherein the cgRNA is configured to be inactivated by the binding of the domain a of the modified effector handle loop region and the domain a* of the input target to each other and the domain b of the terminator insert region and the domain b* of the input target to each other, wherein by a binding of the input target to the cgRNA, the cgRNA ceases to perform a downstream function on the target nucleic acid.

37. The method of claim 33, comprising:
providing a conditional guide RNA (cgRNA) comprising from 5′ to 3′ the target binding region, the effector handle, a terminator insert region comprising a domain a, and a terminator region, wherein the cgRNA is configured to be active; and
conditionally performing a downstream function on the target nucleic acid by providing an input target comprising from 3′ to 5′ a domain a*, and wherein the domain a of the terminator insert region and the domain a* of the input target are complementary to each other, and wherein the cgRNA is configured to be inactivated by the binding of the domain a of the terminator insert region and the domain a* of the input target to each other, wherein by a binding of the input target to the cgRNA, the cgRNA ceases to perform a downstream function on the target nucleic acid.

38. (canceled)

39. The method of claim 26 for conditionally performing a downstream function on a target nucleic acid, wherein the downstream function is selected from the group consisting of activating an expression of the target nucleic acid, silencing an expression of the target nucleic acid, editing the target nucleic acid, and binding the target nucleic acid.

40. The method of claim 39, wherein changing the activity status of the cgRNA results in a conditional increase or a conditional decrease in the downstream function relative to a basal level of a cgRNA-mediated activity on the target nucleic acid.

41. The method of claim 25, wherein an effector handle region of the cgRNA is configured to interact and form a complex with an effector protein selected from the group consisting of Cas9, dCas9, C2C2, Cas13d, any protein fusions or derivatives thereof, any RNA-guided CRISPR effector protein or protein complex, or any protein from a similar pathway.

42. The method of claim 25, wherein the cgRNA comprises one or more chemical modifications that alter one or more of degradation properties, affinity, biological activity, and delivery properties of the cgRNA.

43. The method of claim 42, wherein the one or more chemical modifications is selected from the group consisting of arabino nucleic acids (ANA), locked nucleic acids (LNA), peptide nucleic acids (PNA), phosphoroamidate DNA analogues, phosphorodiamidate morpholino oligomers (PMO), cyclohexene nucleic acids (CeNA), tricycloDNA (tcDNA), bridged nucleic acids (BNA), phosphorothioate modification, 2′-fluoro (2′-F) modification, 2′-fluoroarabino (2′-FANA) modification, 2′O-Methyl (2′O-Me) modification, and 2′O-(2-methoxyethyl) (2′O-MOE) modification.

44. The method of claim 25, wherein a sequence of the cgRNA may be a subsequence of a longer RNA, DNA, or another polymer capable of base-pairing.

45. The method of claim 26, wherein one or more secondary structures formed by the domains of the cgRNA and/or cgRNA-input target complex that are complementary to each other may contain one or more of mismatches, loops, multiloops or bulges due to base-pairing interactions within or between any of the cgRNA domains and input target domains.

46. The method of claim 25, wherein the cgRNA may be expressed in the cells, living organisms or artificial settings in which it interacts with effector, input, and/or target, or may be synthesized exogenously and introduced.

47. The method of claim 25, wherein the cgRNA may conditionally perform a downstream function on a target nucleic acid in one or more of living organisms, ecosystems, tissue extracts, cell lysates, or artificial systems of reconstituted biological components.

48. The method of claim 25, wherein a sequence of input target may be fully constrained, partially constrained, or fully unconstrained by the sequence of target nucleic acid.

49. The method of claim 25, wherein a sequence of input target may be a subsequence of a longer RNA, DNA, or another polymer capable of base-pairing.

50. The method of claim 26, wherein the target nucleic acid may be RNA, DNA, or another polymer capable of base-pairing, coding or non-coding, endogenous or exogenous.

51. The method of claim 26, wherein the RNA-guided effector is selected from the group consisting of Cas9, dCas9, C2C2, Cas13d, protein fusions or derivatives thereof, RNA-guided effector protein or protein complex, any protein from a similar pathway, or any protein the mediates a downstream function on a target nucleic acid in complex with a cgRNA with an active status.

52.-64. (canceled)


SEQUENCE LISTING (32 sequences)

SEQ ID NO: 1 - RNA, 126 nt, Artificial Sequence
  Synthetic
gggauaauaaaagaacaucuaauucaacaagaauuguuuuagagcuacgaacgaaauaaaaguagcaaguuaaaauaaggcuaguccguuaucaacuugaaaaaguggcaccgagucggugcuuuu

SEQ ID NO: 2 - RNA, 30 nt, Artificial Sequence
  Synthetic
cuuuuauuucguucguucuuuuauuauccc

SEQ ID NO: 3 - RNA, 126 nt, Artificial Sequence; identical to SEQ ID NO: 1
  Synthetic

SEQ ID NO: 4 - RNA, 126 nt, Artificial Sequence
  Synthetic
gggcauaaguguagacaucuaauucaacaagaauuguuuuagagcuaauaagaauaauaaaguagcaaguuaaaauaaggcuaguccguuaucaacuugaaaaaguggcaccgagucggugcuuuu

SEQ ID NO: 5 - RNA, 126 nt, Artificial Sequence
  Synthetic
gggaaacagcaacaucaucuaauucaacaagaauuguuuuagagcuauaaagagugaauagguagcaaguuaaaauaaggcuaguccguuaucaacuugaaaaaguggcaccgagucggugcuuuu

SEQ ID NO: 6 - RNA, 126 nt, Artificial Sequence
  Synthetic
gggagaaauuaugaucaucuaauucaacaagaauuguuuuagagcuauggagaaguaagaaguagcaaguuaaaauaaggcuaguccguuaucaacuugaaaaaguggcaccgagucggugcuuuu

SEQ ID NO: 7 - RNA, 30 nt, Artificial Sequence; identical to SEQ ID NO: 2
  Synthetic

SEQ ID NO: 8 - RNA, 30 nt, Artificial Sequence
  Synthetic
cuuuauuauucuuauucuacacuuaugccc

SEQ ID NO: 9 - RNA, 30 nt, Artificial Sequence
  Synthetic
ccuauucacucuuuaauguugcuguuuccc

SEQ ID NO: 10 - RNA, 30 nt, Artificial Sequence
  Synthetic
cuucuuacuucuccaaucauaauuucuccc

SEQ ID NO: 11 - RNA, 164 nt, Artificial Sequence
  Synthetic
caucuaauucaacaagaauuguuuuagagcuagucguuaccuuaucaauaucaaccuccgcauacacuagcaaguuaaaauaaggcuaguccguuaucaacuugcacauaggacccaacaugccaacagagaagaguuaaguggcaccgagucggugcuuuuuu

SEQ ID NO: 12 - RNA, 164 nt, Artificial Sequence
  Synthetic
caucuaauucaacaagaauuguuuuagagcuaguaaucgaaucauaguaaauuucccaucgucauaauagcaaguuaaaauaaggcuaguccguuaucaacuucauacgggucugaaguaguucauucuuauacagucaaguggcaccgagucggugcuuuuuu

SEQ ID NO: 13 - RNA, 164 nt, Artificial Sequence
  Synthetic
caucuaauucaacaagaauuguuuuagagcuacaccuuacgccgguucaauuccaagucccuuccaguagcaaguuaaaauaaggcuaguccguuaucaacuuaacacccuuuacaaaccuuccucuuccuuuacccuaaguggcaccgagucggugcuuuuuu

SEQ ID NO: 14 - RNA, 109 nt, Artificial Sequence
  Synthetic
aacucuucucuguuggcauguuggguccuaugugcguguaugcggagguugauauugauaagguaacgacaaaaaaaaaccccgccccugacagggcgggguuuuuuuu

SEQ ID NO: 15 - RNA, 109 nt, Artificial Sequence
  Synthetic
gacuguauaagaaugaacuacuucagacccguauguuaugacgaugggaaauuuacuaugauucgauuacaaaaaaaaaccccgccccugacagggcgggguuuuuuuu

SEQ ID NO: 16 - RNA, 109 nt, Artificial Sequence
  Synthetic
aggguaaaggaagaggaagguuuguaaaggguguucuggaagggacuuggaauugaaccggcguaaggugaaaaaaaaaccccgccccugacagggcgggguuuuuuuu

SEQ ID NO: 17 - RNA, 128 nt, Artificial Sequence
  Synthetic
aacuuucaguuuagcggucuguuuuagagcuagaaauagcaaguuaaaauaaggcuaguccgaucaaacggguaaacaaacaggauaauuaaggaggcaguacccgggcaccgagucggugcuuuuuu

SEQ ID NO: 18 - RNA, 74 nt, Artificial Sequence
  Synthetic
uacugccuccuuaauuauccuguuuguuuacccguuugaucgcaaaaaaccccgcuucggcgggguuuuuucgc

SEQ ID NO: 19 - RNA, 163 nt, Artificial Sequence
  Synthetic
uuuauggcuagcucaguccuagguacaaugcuagcaacuuucaguuuagcggucuguuuuagagcuagaaauagcaaguuaaaauaaggcuaguccgaauauaggggaagagaaagaagaagagaagagaaagauguccccggcaccgagucggugcuuuuuu

SEQ ID NO: 20 - RNA, 128 nt, Artificial Sequence
  Synthetic
aacuuucaguuuagcggucuguuuuagagcuagaaauagcaaguuaaaauaaggcuaguccguaucaugggguuguguguuguuguaaguguguguguguugccccggcaccgagucggugcuuuuuu

SEQ ID NO: 21 - RNA, 128 nt, Artificial Sequence; identical to SEQ ID NO: 17
  Synthetic

SEQ ID NO: 22 - RNA, 74 nt, Artificial Sequence
  Synthetic
acaucuuucucuucucuucuucuuucucuuccccuauauucgcaaaaaaccccgcuucggcgggguuuuuucgc

SEQ ID NO: 23 - RNA, 74 nt, Artificial Sequence
  Synthetic
caacacacacacacuuacaacaacacacaaccccaugauacgcaaaaaaccccgcuucggcgggguuuuuucgc

SEQ ID NO: 24 - RNA, 74 nt, Artificial Sequence; identical to SEQ ID NO: 18
  Synthetic

SEQ ID NO: 25 - RNA, 130 nt, Artificial Sequence
  Synthetic
agaccgcuaaacugaaaguuacacgcccaacuuucaguuuagcggucuguuuuagagcuagaaauagcaaguuaaaauaaggcuaguccguuaucaacuugaaaaaguggcaccgagucggugcuuuuuu

SEQ ID NO: 26 - RNA, 77 nt, Artificial Sequence
  Synthetic
gggcguguaacuuucaguuuagcggucuacauauuacauuaggcgcaaaaaaccccgcuucggcgggguuuuuucgc

SEQ ID NO: 27 - RNA, 143 nt, Artificial Sequence
  Synthetic
auguucguuguauuaagaccgcuaaacugaaaguuacacgcccaacuuucaguuuagcggucuguuuuagagcuagaaauagcaaguuaaaauaaggcuaguccguuaucaacuugaaaaaguggcaccgagucggugcuuuu

SEQ ID NO: 28 - RNA, 143 nt, Artificial Sequence
  Synthetic
guauaugaaauugaaagaccgcuaaacugaaaguuacacgcccaacuuucaguuuagcggucuguuuuagagcuagaaauagcaaguuaaaauaaggcuaguccguuaucaacuugaaaaaguggcaccgagucggugcuuuu

SEQ ID NO: 29 - RNA, 143 nt, Artificial Sequence
  Synthetic
aaggugauaguaaagagaccgcuaaacugaaaguuacacgcccaacuuucaguuuagcggucuguuuuagagcuagaaauagcaaguuaaaauaaggcuaguccguuaucaacuugaaaaaguggcaccgagucggugcuuuu

SEQ ID NO: 30 - RNA, 68 nt, Artificial Sequence
  Synthetic
aacuuucaguuuagcggucuuaauacaacgaacauaaaaaaaggaucucaagaagauccuuugauuuu

SEQ ID NO: 31 - RNA, 68 nt, Artificial Sequence
  Synthetic
aacuuucaguuuagcggucuuucaauuucauauacaaaaaaaggaucucaagaagauccuuugauuuu

SEQ ID NO: 32 - RNA, 68 nt, Artificial Sequence
  Synthetic
aacuuucaguuuagcggucucuuuacuaucaccuuaaaaaaaggaucucaagaagauccuuugauuuu